SEQUENCE LISTING (64 sequences)

SEQ ID NO: 1 - protein, 60 aa, Sulfolobus solfataricus
MSSGKKPVKVKTPAGKEAELVPEKVWALAPKGRKGVKIGLFKDPETGKYFRHKLPDDYPI

SEQ ID NO: 2 - protein, 59 aa, Sulfolobus acidocaldarius
MSEKKRVRVRTPGGKELELTPEKTWVLAPKGRKGVKIGLFKDPESGKYFRHKLPDDYPV

SEQ ID NO: 3 - protein, 59 aa, Metallosphaera sedula
MTYKKAVKIKTPGGKEAELAPEKAWTLAPKGRKGVKIGLFKDPESGKYFRHKLPDDYPV

SEQ ID NO: 4 - protein, 61 aa, Staphylothermus marinus
MAACKDAVKVKTLSGKEVELVPKKVWQLSPKGRKGVKVGLFQDPETGKYFAKVPDDYPICG

SEQ ID NO: 5 - protein, 61 aa, Hyperthermus butylicus
MACEKPVKVRDPTTGKEVELVPIKVWQLAPKGRKGVKIGLFKSPETGKYFAKVPDDYPICS

SEQ ID NO: 6 - protein, 62 aa, Hyperthermus butylicus
MACEKPVKVRDPTTGKEVELVPIKVWQLAPRGRKGVKIGLFKSPETGKYFRAKVPDDYPICS

SEQ ID NO: 7 - protein, 63 aa, Aeropyrum pernix
MSQKQLPPVKVRDPTTGKEVELTPIKVWKLSPRGRRGVKIGLFKSPETGKFRAKVPDDYPETG

SEQ ID NO: 8 - protein, 76 aa, Caldivirga maquilingensis
MLFMFISHYAVYLLTGMAVNVQQYLNKEYEVECDGQMVRLKPVKAWVLQPGRKGVVIGLFKCPNGKTLRKAIGKIE

SEQ ID NO: 9 - protein, 55 aa, Ignicoccus hospitalis
MPKCPKCGAEVKEPIKTWVLAPKGRKGVIIGLFRCPNGHYFRAKVGEAPPKKEAA

SEQ ID NO: 10 - protein, 59 aa, Pyrobaculum islandicum
MEEVLDREYEVEYGGRKYRLKPVKAWVLQPPGKPGVVIALFKLPDGKTIRKVIMKLPPS

SEQ ID NO: 11 - protein, 59 aa, Pyrobaculum arsenaticum
MAEEILNREYEVEYGGKRYILRPIKAWVLQPPGKPGVVVALFRLPDGKTVRKVVMKLPP

SEQ ID NO: 12 - protein, 59 aa, Pyrobaculum aerophilum
MAEEILNREYEVEYEGRKYFLRPVKAWVLQPPGKPGVVVALFKLPNGKSIRKVIMRLPP

SEQ ID NO: 13 - protein, 63 aa, Pyrobaculum calidifontis
MDQDVAEEILNKEYEVVYEGKRFLLKPAKAWVLQPPGKPGVIVALFKLPNGKTVRKVIARLPP

SEQ ID NO: 14 - protein, 59 aa, Thermoproteus neutrophilus
MAEEILNREYEVEYGGKRYWLRPSKAWVLQPPGKPGVVIALFKLPDGRTVRKAIMRLPP

SEQ ID NO: 15 - protein, 40 aa, Artificial Sequence
  Consensus sequence
GXXXXXXPXKXWXLXPXGXXGVXXXLFXXPXXGXXXRXXX

SEQ ID NO: 16 - protein, 621 aa, Chimeric protein
MSQKQLPPVKVRDPTTGKEVELTPIKVWKLSPRGRRGVKIGLFKSPETGKYFRAKVPDDYPETGGTHMGLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 17 - protein, 901 aa, Artificial Sequence
  Chimeric protein
MSQKQLPPVKVRDPTTGKEVELTPIKVWKLSPRGRRGVKIGLFKSPETGKYFRAKVPDDYPETGGGVTMDSGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 18 - protein, 841 aa, Artificial Sequence
  Chimeric protein
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKSGTHSQKQLPPVKVRDPTTGKEVELTPIKVWKLSPRGRRGVKIGLFKSPETGKYFRAKVPDDYPETG

SEQ ID NO: 19 - protein, 617 aa, Artificial Sequence
  Chimeric protein
MSSGKKPVKVKTPAGKEAELVPEKVWALAPKGRKGVKIGLFKDPETGKYFRHKLPDDYPIGTHMGLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 20 - protein, 897 aa, Artificial Sequence
  Chimeric protein
MSSGKKPVKVKTPAGKEAELVPEKVWALAPKGRKGVKIGLFKDPETGKYFRHKLPDDYPIGGVTMDSGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 21 - protein, 838 aa, Artificial Sequence
  Chimeric protein
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKSGTHMSSGKKPVKVKTPAGKEAELVPEKVWALAPKGRKGVKIGLFKDPETGKYFRHKLPDDYPI

SEQ ID NO: 22 - protein, 616 aa, Artificial Sequence
  Chimeric protein
MEEVLDREYEVEYGGRKYRLKPVKAWVLQPPGKPGVVIALFKLPDGKTIRKVIMKLPPSVTHMGLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 23 - protein, 899 aa, Artificial Sequence
  Chimeric protein
MACEKPVKVRDPTTGKEVELVPIKVWQLAPRGRKGVKIGLFKSPETGKYFRAKVPDDYPICSGGVTMDSGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 24 - protein, 840 aa, Artificial Sequence
  Chimeric protein
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKSGTHMACEKPVKVRDPTTGKEVELVPIKVWQLAPRGRKGVKIGLFKSPETGKYFRAKVPDDYPICS

SEQ ID NO: 25 - DNA, 1866 bp, Artificial Sequence
  cloned construct
atgagccagaagcaactaccacctgtgaaggtcagggacccgactacaggcaaggaggtcgagctaacgccaatcaaagtgtggaagctatcgccgagggggaggaggggcgtcaagataggtctcttcaagagccccgagacgggcaagtacttcagggccaaggtgcccgacgactaccccgagaccgggggtacccatatgggtctgctgcacgaattcggtctgctggaatctccgaaagcgctggaagaagcgccgtggccgccgccggaaggtgcgttcgttggtttcgttctgtctcgtaaagaaccgatgtgggcggacctgctggcgctggcggcggcgcgtggtggtcgtgttcaccgtgcgccggaaccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 26 - DNA, 2706 bp, Artificial Sequence
  cloned construct
atgagccagaagcaactaccacctgtgaaggtcagggacccgactacaggcaaggaggtcgagctaacgccaatcaaagtgtggaagctatcgccgagggggaggaggggcgtcaagataggtctcttcaagagccccgagacgggcaagtacttcagggccaaggtgcccgacgactaccccgagaccgggggtggtgttaccatgaattcggggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 27 - DNA, 2526 bp, Artificial Sequence
  cloned construct
atgattttagatgtggattacataactgaagaaggaaaacctgttattaggctattcaaaaaagagaacggaaaatttaagatagagcatgatagaacttttagaccatacatttacgctcttctcagggatgattcaaagattgaagaagttaagaaaataacgggggaaaggcatggaaagattgtgagaattgttgatgtagagaaggttgagaaaaagtttctcggcaagcctattaccgtgtggaaactttatttggaacatccccaagatgttcccactattagagaaaaagttagagaacatccagcagttgtggacatcttcgaatacgatattccatttgcaaagagatacctcatcgacaaaggcctaataccaatggagggggaagaagagctaaagattcttgccttcgatatagaaaccctctatcacgaaggagaagagtttggaaaaggcccaattataatgattagttatgcagatgaaaatgaagcaaaggtgattacttggaaaaacatagatcttccatacgttgaggttgtatcaagcgagagagagatgataaagagatttctcaggattatcagggagaaggatcctgacattatagttacttataatggagactcattcgacttcccatatttagcgaaaagggcagaaaaacttgggattaaattaaccattggaagagatggaagcgagcccaagatgcagagaataggcgatatgacggctgtagaagtcaagggaagaatacatttcgacttgtatcatgtaataacaaggacaataaatctcccaacatacacactagaggctgtatatgaagcaatttttggaaagccaaaggagaaggtatacgccgacgagatagcaaaagcctgggaaagtggagagaaccttgagagagttgccaaatactcgatggaagatgcaaaggcaacttatgaactcgggaaagaattccttccaatggaaattcagctttcaagattagttggacaacctttatgggatgtttcaaggtcaagcacagggaaccttgtagagtggttcttacttaggaaagcctacgaaagaaacgaagtagctccaaacaagccaagtgaagaggagtatcaaagaaggctcagggagagctacacaggtggattcgttaaagagccagaaaaggggttgtgggaaaacatagtatacctagattttagagccctatatccctcgattataattacccacaatgtttctcccgatactctaaatcttgagggatgcaagaactatgatatcgctcctcaagtaggccacaagttctgcaaggacatccctggttttataccaagtctcttgggacatttgttagaggaaagacaaaagattaagacaaaaatgaaggaaactcaagatcctatagaaaaaatactccttgactatagacaaaaagcgataaaactcttagcaaattctttctacggatattatggctatgcaaaagcaagatggtactgtaaggagtgtgctgagagcgttactgcctggggaagaaagtacatcgagttagtatggaaggagctcgaagaaaagtttggatttaaagtcctctacattgacactgatggtctctatgcaactatcccaggaggagaaagtgaggaaataaagaaaaaggctctagaatttgtaaaatacataaattcaaagctccctggactgctagagcttgaatatgaagggttttataagaggggattcttcgttacgaagaagaggtatgcagtaatagatgaagaaggaaaagtcattactcgtggtttagagatagttaggagagattggagtgaaattgcaaaagaaactcaagctagagttttggagacaatactaaaacacggagatgttgaagaagctgtgagaatagtaaaagaagtaatacaaaagcttgccaattatgaaattccaccagagaagctcgcaatatatgagcagataacaagaccattacatgagtataaggcgataggtcctcacgtagctgttgcaaagaaactagctgctaaaggagttaaaataaagccaggaatggtaattggatacatagtacttagaggcgatggtccaattagcaatagggcaattctagctgaggaatacgatcccaaaaagcacaagtatgacgcagaatattacattgagaaccaggttcttccagcggtacttaggatattggagggatttggatacagaaaggaagacctcagataccaaaagacaagacaagtcggcctaacttcctggcttaacattaaaaaatccggtacccatagccagaagcaactaccacctgtgaaggtcagggacccgactacaggcaaggaggtcgagctaacgccaatcaaagtgtggaagctatcgccgagggggaggaggggcgtcaagataggtctcttcaagagccccgagacgggcaagtacttcagggccaaggtgcccgacgactaccccgagaccgggtaa

SEQ ID NO: 28 - DNA, 1854 bp, Artificial Sequence
  cloned construct
atgagttcgggtaaaaaaccagtaaaagtaaaaacaccagctggtaaagaggctgaattggttccagaaaaagtatgggcattagcaccaaagggtagaaaaggtgtaaagataggtttatttaaagatccagaaactgggaaatacttcagacataagctaccagatgattatccaataggtacccatatgggtctgctgcacgaattcggtctgctggaatctccgaaagcgctggaagaagcgccgtggccgccgccggaaggtgcgttcgttggtttcgttctgtctcgtaaagaaccgatgtgggcggacctgctggcgctggcggcggcgcgtggtggtcgtgttcaccgtgcgccggaaccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 29 - DNA, 2694 bp, Artificial Sequence
  cloned construct
atgagttcgggtaaaaaaccagtaaaagtaaaaacaccagctggtaaagaggctgaattggttccagaaaaagtatgggcattagcaccaaagggtagaaaaggtgtaaagataggtttatttaaagatccagaaactgggaaatacttcagacataagctaccagatgattatccaataggtggtgttaccatgaattcggggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 30 - DNA, 2517 bp, Artificial Sequence
  cloned construct
atgattttagatgtggattacataactgaagaaggaaaacctgttattaggctattcaaaaaagagaacggaaaatttaagatagagcatgatagaacttttagaccatacatttacgctcttctcagggatgattcaaagattgaagaagttaagaaaataacgggggaaaggcatggaaagattgtgagaattgttgatgtagagaaggttgagaaaaagtttctcggcaagcctattaccgtgtggaaactttatttggaacatccccaagatgttcccactattagagaaaaagttagagaacatccagcagttgtggacatcttcgaatacgatattccatttgcaaagagatacctcatcgacaaaggcctaataccaatggagggggaagaagagctaaagattcttgccttcgatatagaaaccctctatcacgaaggagaagagtttggaaaaggcccaattataatgattagttatgcagatgaaaatgaagcaaaggtgattacttggaaaaacatagatcttccatacgttgaggttgtatcaagcgagagagagatgataaagagatttctcaggattatcagggagaaggatcctgacattatagttacttataatggagactcattcgacttcccatatttagcgaaaagggcagaaaaacttgggattaaattaaccattggaagagatggaagcgagcccaagatgcagagaataggcgatatgacggctgtagaagtcaagggaagaatacatttcgacttgtatcatgtaataacaaggacaataaatctcccaacatacacactagaggctgtatatgaagcaatttttggaaagccaaaggagaaggtatacgccgacgagatagcaaaagcctgggaaagtggagagaaccttgagagagttgccaaatactcgatggaagatgcaaaggcaacttatgaactcgggaaagaattccttccaatggaaattcagctttcaagattagttggacaacctttatgggatgtttcaaggtcaagcacagggaaccttgtagagtggttcttacttaggaaagcctacgaaagaaacgaagtagctccaaacaagccaagtgaagaggagtatcaaagaaggctcagggagagctacacaggtggattcgttaaagagccagaaaaggggttgtgggaaaacatagtatacctagattttagagccctatatccctcgattataattacccacaatgtttctcccgatactctaaatcttgagggatgcaagaactatgatatcgctcctcaagtaggccacaagttctgcaaggacatccctggttttataccaagtctcttgggacatttgttagaggaaagacaaaagattaagacaaaaatgaaggaaactcaagatcctatagaaaaaatactccttgactatagacaaaaagcgataaaactcttagcaaattctttctacggatattatggctatgcaaaagcaagatggtactgtaaggagtgtgctgagagcgttactgcctggggaagaaagtacatcgagttagtatggaaggagctcgaagaaaagtttggatttaaagtcctctacattgacactgatggtctctatgcaactatcccaggaggagaaagtgaggaaataaagaaaaaggctctagaatttgtaaaatacataaattcaaagctccctggactgctagagcttgaatatgaagggttttataagaggggattcttcgttacgaagaagaggtatgcagtaatagatgaagaaggaaaagtcattactcgtggtttagagatagttaggagagattggagtgaaattgcaaaagaaactcaagctagagttttggagacaatactaaaacacggagatgttgaagaagctgtgagaatagtaaaagaagtaatacaaaagcttgccaattatgaaattccaccagagaagctcgcaatatatgagcagataacaagaccattacatgagtataaggcgataggtcctcacgtagctgttgcaaagaaactagctgctaaaggagttaaaataaagccaggaatggtaattggatacatagtacttagaggcgatggtccaattagcaatagggcaattctagctgaggaatacgatcccaaaaagcacaagtatgacgcagaatattacattgagaaccaggttcttccagcggtacttaggatattggagggatttggatacagaaaggaagacctcagataccaaaagacaagacaagtcggcctaacttcctggcttaacattaaaaaatccggtacccatatgagttcgggtaaaaaaccagtaaaagtaaaaacaccagctggtaaagaggctgaattggttccagaaaaagtatgggcattagcaccaaagggtagaaaaggtgtaaagataggtttatttaaagatccagaaactgggaaatacttcagacataagctaccagatgattatccaatataa

SEQ ID NO: 31 - DNA, 1851 bp, Artificial Sequence
  cloned construct
atggaagaggtcttagatcgtgaatacgaagtggaatacggcgggagaaaataccggctaaagccagttaaagcatgggttctccagccccctggcaaaccaggtgtcgtcatagccctctttaaactaccagatggaaaaactattaggaaggtgataatgaaattgccgcctagcgttacccatatgggtctgctgcacgaattcggtctgctggaatctccgaaagcgctggaagaagcgccgtggccgccgccggaaggtgcgttcgttggtttcgttctgtctcgtaaagaaccgatgtgggcggacctgctggcgctggcggcggcgcgtggtggtcgtgttcaccgtgcgccggaaccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 32 - DNA, 2700 bp, Artificial Sequence
  cloned construct
atggcgtgtgagaagcctgttaaggttcgtgaccctactactggtaaggaggtagagctggtaccaatcaaggtgtggcagctagcacccaggggtaggaagggcgtcaagataggcctattcaagagccccgaaacaggcaagtacttcagagccaaggtaccagacgactacccaatctgcagcggtggtgttaccatgaattcggggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 33 - DNA, 2523 bp, Artificial Sequence
  cloned construct
atgattttagatgtggattacataactgaagaaggaaaacctgttattaggctattcaaaaaagagaacggaaaatttaagatagagcatgatagaacttttagaccatacatttacgctcttctcagggatgattcaaagattgaagaagttaagaaaataacgggggaaaggcatggaaagattgtgagaattgttgatgtagagaaggttgagaaaaagtttctcggcaagcctattaccgtgtggaaactttatttggaacatccccaagatgttcccactattagagaaaaagttagagaacatccagcagttgtggacatcttcgaatacgatattccatttgcaaagagatacctcatcgacaaaggcctaataccaatggagggggaagaagagctaaagattcttgccttcgatatagaaaccctctatcacgaaggagaagagtttggaaaaggcccaattataatgattagttatgcagatgaaaatgaagcaaaggtgattacttggaaaaacatagatcttccatacgttgaggttgtatcaagcgagagagagatgataaagagatttctcaggattatcagggagaaggatcctgacattatagttacttataatggagactcattcgacttcccatatttagcgaaaagggcagaaaaacttgggattaaattaaccattggaagagatggaagcgagcccaagatgcagagaataggcgatatgacggctgtagaagtcaagggaagaatacatttcgacttgtatcatgtaataacaaggacaataaatctcccaacatacacactagaggctgtatatgaagcaatttttggaaagccaaaggagaaggtatacgccgacgagatagcaaaagcctgggaaagtggagagaaccttgagagagttgccaaatactcgatggaagatgcaaaggcaacttatgaactcgggaaagaattccttccaatggaaattcagctttcaagattagttggacaacctttatgggatgtttcaaggtcaagcacagggaaccttgtagagtggttcttacttaggaaagcctacgaaagaaacgaagtagctccaaacaagccaagtgaagaggagtatcaaagaaggctcagggagagctacacaggtggattcgttaaagagccagaaaaggggttgtgggaaaacatagtatacctagattttagagccctatatccctcgattataattacccacaatgtttctcccgatactctaaatcttgagggatgcaagaactatgatatcgctcctcaagtaggccacaagttctgcaaggacatccctggttttataccaagtctcttgggacatttgttagaggaaagacaaaagattaagacaaaaatgaaggaaactcaagatcctatagaaaaaatactccttgactatagacaaaaagcgataaaactcttagcaaattctttctacggatattatggctatgcaaaagcaagatggtactgtaaggagtgtgctgagagcgttactgcctggggaagaaagtacatcgagttagtatggaaggagctcgaagaaaagtttggatttaaagtcctctacattgacactgatggtctctatgcaactatcccaggaggagaaagtgaggaaataaagaaaaaggctctagaatttgtaaaatacataaattcaaagctccctggactgctagagcttgaatatgaagggttttataagaggggattcttcgttacgaagaagaggtatgcagtaatagatgaagaaggaaaagtcattactcgtggtttagagatagttaggagagattggagtgaaattgcaaaagaaactcaagctagagttttggagacaatactaaaacacggagatgttgaagaagctgtgagaatagtaaaagaagtaatacaaaagcttgccaattatgaaattccaccagagaagctcgcaatatatgagcagataacaagaccattacatgagtataaggcgataggtcctcacgtagctgttgcaaagaaactagctgctaaaggagttaaaataaagccaggaatggtaattggatacatagtacttagaggcgatggtccaattagcaatagggcaattctagctgaggaatacgatcccaaaaagcacaagtatgacgcagaatattacattgagaaccaggttcttccagcggtacttaggatattggagggatttggatacagaaaggaagacctcagataccaaaagacaagacaagtcggcctaacttcctggcttaacattaaaaaatccggtacccatatggcgtgtgagaagcctgttaaggttcgtgaccctactactggtaaggaggtagagctggtaccaatcaaggtgtggcagctagcacccaggggtaggaagggcgtcaagataggcctattcaagagccccgaaacaggcaagtacttcagagccaaggtaccagacgactacccaatctgcagctaa

SEQ ID NO: 34 - DNA, 30 bp, Artificial Sequence
  Primer sequence
gatatccatatgagccagaagcaactacca

SEQ ID NO: 35 - DNA, 38 bp, Artificial Sequence
  Primer sequence
gaattccatatgggtacccccggtctcggggtagtcgt

SEQ ID NO: 36 - DNA, 29 bp, Artificial Sequence
  Primer sequence
gatatccatatgagttcgggtaaaaaacc

SEQ ID NO: 37 - DNA, 38 bp, Artificial Sequence
  Primer sequence
gaattccatatgggtacctattggataatcatctggta

SEQ ID NO: 38 - DNA, 30 bp, Artificial Sequence
  Primer sequence
gatatccatatggaagaggtcttagatcgt

SEQ ID NO: 39 - DNA, 38 bp, Artificial Sequence
  Priemr sequence
gaattccatatgggtaacgctaggcggcaatttcatta

SEQ ID NO: 40 - DNA, 45 bp, Artificial Sequence
  Primer sequence
atccccgaattcatggtaacaccacccccggtctcggggtagtcg

SEQ ID NO: 41 - protein, 4 aa, Artificial Sequence
  Linker peptide
GGVT

SEQ ID NO: 42 - DNA, 45 bp, Artificial Sequence
  Primer sequence
atccccgaattcatggtaacaccacctattggataatcatctggt

SEQ ID NO: 43 - DNA, 30 bp, Artificial Sequence
  Primer sequence
gatatccatatggcgtgtgagaagcctgtt

SEQ ID NO: 44 - DNA, 45 bp, Artificial Sequence
  Primer sequence
atccccgaattcatggtaacaccaccgctgcagattgggtagtcg

SEQ ID NO: 45 - DNA, 30 bp, Artificial Sequence
  Primer sequence
gaattcggtacccatagccagaagcaacta

SEQ ID NO: 46 - DNA, 30 bp, Artificial Sequence
  Primer sequence
gaattcgtcgacttacccggtctcggggta

SEQ ID NO: 47 - DNA, 30 bp, Artificial Sequence
  Primer sequence
gaattcggtacccatatgagttcgggtaaa

SEQ ID NO: 48 - DNA, 30 bp, Artificial Sequence
  Primer sequence
gaattcgtcgacttatattggataatcatc

SEQ ID NO: 49 - DNA, 30 bp, Artificial Sequence
  Primer sequence
gaattcggtacccatatggcgtgtgagaag

SEQ ID NO: 50 - DNA, 30 bp, Artificial Sequence
  Primer sequence
gaattcgtcgacttagctgcagattgggta

SEQ ID NO: 51 - DNA, 20 bp, Artificial Sequence
  Primer sequence
cctgctctgccgcttcacgc

SEQ ID NO: 52 - DNA, 28 bp, Artificial Sequence
  Primer sequence
tccggataaaaacgtcgatgacatttgc

SEQ ID NO: 53 - DNA, 28 bp, Artificial Sequence
  Primer sequence
gatgacgcatcctcacgataatatccgg

SEQ ID NO: 54 - DNA, 24 bp, Artificial Sequence
  Primer sequence
ccatgattcagtgtgcccgtctgg

SEQ ID NO: 55 - DNA, 23 bp, Artificial Sequence
  Primer sequence
cgaacgtcgcgcagagaaacagg

SEQ ID NO: 56 - DNA, 23 bp, Artificial Sequence
  Primer sequence
gcctcgttgcgtttgtttgcacg

SEQ ID NO: 57 - DNA, 27 bp, Artificial Sequence
  Primer sequence
gcacagaagctattatgcgtccccagg

SEQ ID NO: 58 - DNA, 26 bp, Artificial Sequence
  Primer sequence
tcttcctcgtgcatcgagctattcgg

SEQ ID NO: 59 - protein, 60 aa, Sulfolobus shibatae
MSSGKKAVKVKTPAGKEAELVPEKVWALAPKGRKGVKIGLFKDPETGKYFRHKLPDDYPI

SEQ ID NO: 60 - protein, 57 aa, Sulfolobus tokodaii
MAEKKVKVKTPSGKEAELAPEKVWVLAPKGRKGVKIGLFKDPETGKYFRHKLPDDYP

SEQ ID NO: 61 - protein, 45 aa, Artificial Sequence
  Consensus sequence
XXXXXGXXXXXXPXKXWXLXPXGXXGVXXXLFXXPXXGXXXRXXX

SEQ ID NO: 62 - DNA, 28 bp, Artificial Sequence
  Primer sequence
ccactgtaaagctaacttagcattaacc

SEQ ID NO: 63 - DNA, 28 bp, Artificial Sequence
  Primer sequence
gtgatgaggaatagtgtaaggagtatgg

SEQ ID NO: 64 - DNA, 29 bp, Artificial Sequence
  PCR probe sequence
ccaacacctctttacagtgaaatgcccca

(19) United States
(12) Patent Application Publication
(10) Pub. No.: US 2011/0020877 A1
(43) Pub. Date: Jan. 27, 2011

(54) CREN7 CHIMERIC PROTEIN

(75) Inventors: Duncan Roy Clark, Surrey (GB); Martin Wilkinson, Surrey (GB); Nicholas Morant, Surrey (GB)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Genesys Limited, Camberley, Surrey (GB)

(21) Appl. No.: 12/812,519

(22) PCT Filed: Jan. 9, 2009
(86) PCT No.: PCT/GB09/00063

§ 371 Date: Oct. 14, 2010

Related U.S. Application Data
(60) Provisional application No. 61/010,812, filed on Jan. 11, 2008.

(51) Int. Cl. C12P 19/34; C12N 9/12; C07H 21/00; C12N 15/63; C12N 1/21

(52) U.S. Cl. 435/91.2; 435/194; 536/23.2; 435/320.1; 435/252.33; 435/91.1

(57) ABSTRACT

There is provided a chimeric protein comprising a nucleic acid modifying enzyme domain having nucleic acid modifying activity joined with an Cren7 enhancer domain or variant thereof, in which the Cren7 enhancer domain or variant thereof enhances the activity of the nucleic acid modifying enzyme domain compared with a corresponding protein lacking the Cren7 enhancer domain or variant thereof. There is also provided an isolated nucleic acid encoding the chimeric protein of the invention and methods utilising the protein.

### FIELD OF INVENTION

The present invention relates to improved nucleic acid modifying enzymes such as nucleic acid polymerases, and their use.

### BACKGROUND

Nucleic acid modifying enzymes, especially thermostable DNA polymerases, have become an important tool for the molecular biologist. Various approaches for enhancing the activity of naturally-found DNA polymerases have been reported. For example, Motz et al. (2002, J. Biol. Chem. 277: 16179-16188) found that a proliferating cell nuclear antigen homologue from Archaeoglobus fulgidus, when fused to the classical PCR enzyme Taq DNA polymerase from Thermus aquaticus, stimulated processivity of the DNA polymerase. Davidson et al. (2003, Nucleic Acids Res. 31: 4702-4709) inserted the T3 DNA polymerase thioredoxin binding domain into the distantly related Taq DNA polymerase and showed that this improved the processivity and fidelity of the Taq DNA polymerase. Wang et al. (2004, Nucleic Acids Res. 32: 1197-1207; see also WO01/92501, WO2004/037979 and US2007/0141591) improved the processivity of DNA polymerases (or mutants thereof) by fusing the polymerase domain to a double-stranded DNA binding protein Sso7d from Sulfolobus solfataricus, Sso7d-like proteins, or mutants thereof. Meanwhile, Pavlov et al. (2002, Proc. Natl. Acad. Sci. USA 99: 13510-13515) taught that fusion of a number of helix-hairpin-helix motifs derived from DNA topoisomerase V to each of various DNA polymerases conferred salt resistance and increased processivity.

The present invention provides an alternative approach to enhancing nucleic acid modifying enzyme activity.

### SUMMARY OF INVENTION

According to the present invention there is provided a chimeric protein comprising or consisting essentially of a nucleic acid modifying enzyme domain having nucleic acid modifying activity joined with a Cren7 enhancer domain or a variant thereof, in which the Cren7 enhancer domain enhances the activity of the nucleic acid modifying enzyme domain compared with a corresponding protein lacking the Cren7 enhancer domain (i.e., an identical protein comprising the nucleic acid modifying enzyme domain but lacking the Cren7 enhancer domain).

The Cren7 protein family is highly conserved in Crenarchael organisms, as suggested recently in Guo et al. (2007, Nucleic Acids Research Advance Access published 20 December 2007, 1-9). These workers suggested that the protein of SEQ ID NO: 1 below (labelled by them as “Cren7”) is a putative major chromatin protein with a function in DNA supercoiling and compaction. The inventors have surprisingly found that this domain is useful to enhance the properties of a nucleic acid modifying enzyme domain and there was no suggestion of this in the publication by Guo et al.

This domain classification has been adopted as standard in the art for use with databases of protein sequences, such that the skilled person is able to search such databases, using a known Cren7 sequence such as SEQ ID NO:1 (for example by means of a BLASTP homology search) to determine whether a given organism expresses or is capable of expressing a Cren7 protein. See, for example:

http://expasy.org/cgi-bin/get-similar?name=Cren7%20family;

http://www.uniprot.org/uniprot/?query=family:%22Cren7+family%22; or

http://www.uniprot.org/uniprot/Q97ZE3 (all accessed on 6 January 2009).

Therefore, according to the present invention, the Cren7 enhancer domain is preferably a Cren7 enhancer protein from a Crenarchaeal organism (i.e., an organism from the Phylum Crenarchaeota), which may be selected from an organism within the Class Thermoprotei. The organism may be in the Order Thermoproteales (such as Caldivirga maquilingensis; Pyrobaculum islandicum; Pyrobaculum arsenaticum; Pyrobaculum aerophilum; Pyrobaculum calidifontis; and Therinoproteus neutrophilus), or within the Order Sulfolobales (such as Sulfolobus solfataricus; Sulfolobus acidocaldarius; Sulfolobus shibatae; Sulfolobus tokodaii; and Metallosphaera sedula) or within the Order Desulfurococcales (such as Staphylothermus marinus; Hyperthermus butylicus; Aeropyrum pernix; and Ignicoccus hospitalis).

The variant of the Cren7 enhancer domain, as encompassed by the invention, may be a structural variant, for example, having a percentage sequence similarity to a known Cren7 enhancer protein as set out in Tables 1 and 2 below, determined using the MatGAT program and the alignment matrix BLOSUM50 (Table 1) or BLOSUM62 (Table 2). MatGAT is a type of sequence comparison software which does not rely on comparison to a single sequence but can compare all similar proteins within a specified group. The software has been described by Campanella et al. (BMC Bioinformatics (2003) 4: 29) and is available at http://vvww.bitincka.com/ledion/matgat/ (as accessed on 6 Jan. 2009). For example, the Cren7 enhancer domain may have at least 29% sequence identity to SEQ ID NO:13 (an example of a Cren7 enhancer domain for use according to this invention), determined using the MatGAT program and the alignment matrix BLOSUM62 (see Table 2), or at least 33% sequence identity to SEQ ID NO:1 (another example of a Cren7 enhancer domain for use according to this invention), determined using the MatGAT program and the alignment matrix BLOSUM50 (see Table 1). When using either alignment matrix as referred to throughout this specification, the default program parameters were used, namely, First Gap 12, Extending Gap 2.

In an alternative determination, the variant of the Cren7 enhancer domain may be a structural variant, for example, having at least 35% sequence identity to the Sulfolobus solfataricus Cren7 enhancer protein (SEQ ID NO: 1) when determined using BLASTP with SEQ ID NO:1 as the base sequence. The Cren7 enhancer domain may, for example, have at least 40%, 45%, 50%, 55%, 60%, 65%, 70%, 75%, 80%, 85%, 90%, 95%, 96%, 97%, 98% or even 99% sequence identity, to the Sulfolobus solfataricus Cren7 enhancer protein (SEQ ID NO: 1), when determined using BLASTP with SEQ ID NO:1 as the base sequence. This means that SEQ ID NO:1 is the sequence against which the percentage identity is determined. The BLAST software is publicly available at http://blast.ncbi.nlm.nih.gov/Blast.cgi (accessible on 6 Jan. 2009). Different levels of percentage identity may be determined when using the BLASTP software if a sequence other than SEQ ID NO:1 is used as the base sequence.

TABLE 1
MatGAT comparison of sequences SEQ ID NO: 1-14, 59 & 60
(BLOSUM50 alignment matrix)
Light grey shows % identity; dark grey shows % similarity.

| | | 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|---|---|
| 1 | P_islandicum | | 78.3 | 65.6 | 81.7 | 35.9 | 33.9 | 33.9 | 39.1 | 42.2 | 37.5 | 31.7 | 78.3 | 30.6 | 37.5 | 39.7 | 57.1 |
| | (SEQ ID NO: 10) | | | | | | | | | | | | | | | | |
| 2 | P_arsenaticum | 96.6 | | 73.0 | 81.4 | 35.9 | 38.1 | 38.1 | 35.5 | 39.3 | 33.9 | 32.8 | 86.4 | 35.9 | 33.9 | 40.6 | 51.6 |
| | (SEQ ID NO: 11) | | | | | | | | | | | | | | | | |
| 3 | P_calidifontis | 84.1 | 87.3 | | 74.6 | 34.3 | 32.8 | 32.8 | 36.9 | 36.4 | 34.8 | 32.4 | 73.0 | 34.3 | 34.8 | 38.5 | 54.0 |
| | (SEQ ID NO: 13) | | | | | | | | | | | | | | | | |
| 4 | P_aerophilum | 94.9 | 96.6 | 88.9 | | 34.4 | 36.5 | 36.5 | 33.9 | 38.7 | 33.9 | 29.7 | 81.4 | 35.9 | 33.9 | 37.7 | 54.8 |
| | (SEQ ID NO: 12) | | | | | | | | | | | | | | | | |
| 5 | A_permix | 48.4 | 48.4 | 43.8 | 48.4 | | 78.1 | 79.7 | 59.4 | 64.1 | 65.6 | 45.5 | 32.8 | 65.6 | 64.1 | 64.1 | 35.8 |
| | (SEQ ID NO: 7) | | | | | | | | | | | | | | | | |
| 6 | H_buytlicus_0878 | 45.2 | 48.4 | 44.4 | 48.4 | 89.1 | | 98.4 | 66.1 | 67.7 | 74.6 | 50.0 | 34.9 | 76.2 | 73.0 | 74.2 | 39.7 |
| | (SEQ ID NO: 5) | | | | | | | | | | | | | | | | |
| 7 | H_buytlicus_1128 | 45.2 | 48.4 | 44.4 | 48.4 | 89.1 | 100.0 | | 64.5 | 66.1 | 73.0 | 48.4 | 34.9 | 74.6 | 71.4 | 72.6 | 38.2 |
| | (SEQ ID NO: 6) | | | | | | | | | | | | | | | | |
| 8 | M_sedula | 55.9 | 54.2 | 46.0 | 54.2 | 73.4 | 75.8 | 75.8 | | 83.1 | 81.7 | 43.8 | 35.5 | 66.1 | 83.3 | 84.7 | 38.5 |
| | (SEQ ID NO: 3) | | | | | | | | | | | | | | | | |
| 9 | S_acidocaldarius | 54.2 | 52.5 | 47.6 | 54.2 | 78.1 | 79.0 | 79.0 | 89.8 | | 80.0 | 44.4 | 39.3 | 62.9 | 80.0 | 83.1 | 40.0 |
| | (SEQ ID NO: 2) | | | | | | | | | | | | | | | | |
| 10 | S_solfataricus | 51.7 | 50.0 | 42.9 | 50.0 | 75.0 | 82.3 | 82.3 | 88.3 | 86.7 | | 43.8 | 33.9 | 72.6 | 98.3 | 85.0 | 38.5 |
| | (SEQ ID NO: 1) | | | | | | | | | | | | | | | | |
| 11 | I_hospital | 52.5 | 50.8 | 44.4 | 49.2 | 57.8 | 59.7 | 59.7 | 55.9 | 61.0 | 53.3 | | 31.3 | 43.8 | 41.5 | 46.0 | 38.8 |
| | (SEQ ID NO: 9) | | | | | | | | | | | | | | | | |
| 12 | T_neutrophilus | 93.2 | 94.9 | 85.7 | 94.9 | 45.3 | 45.2 | 45.2 | 52.5 | 50.8 | 48.3 | 45.8 | | 31.3 | 33.9 | 39.3 | 54.8 |
| | (SEQ ID NO: 14) | | | | | | | | | | | | | | | | |
| 13 | S_marinus | 41.9 | 45.2 | 42.9 | 46.8 | 79.7 | 87.1 | 87.1 | 79.0 | 80.6 | 85.5 | 53.2 | 45.2 | | 74.2 | 72.6 | 36.4 |
| | (SEQ ID NO: 4) | | | | | | | | | | | | | | | | |
| 14 | S_shibatae | 51.7 | 50.0 | 42.9 | 50.0 | 73.4 | 80.6 | 80.6 | 90.0 | 86.7 | 98.3 | 51.7 | 48.3 | 87.1 | | 85.0 | 38.5 |
| | (SEQ ID NO: 59) | | | | | | | | | | | | | | | | |
| 15 | S_tokodaii | 55.9 | 57.6 | 47.6 | 54.2 | 78.1 | 79.0 | 79.0 | 88.1 | 89.8 | 90.0 | 57.9 | 54.2 | 80.6 | 90.0 | | 43.8 |
| | (SEQ ID NO: 60) | | | | | | | | | | | | | | | | |
| 16 | C_maquilingensis | 73.8 | 73.8 | 71.4 | 75.4 | 53.1 | 51.6 | 51.6 | 52.5 | 52.5 | 50.8 | 57.4 | 73.8 | 46.8 | 50.8 | 54.1 | |
| | (SEQ ID NO: 8) | | | | | | | | | | | | | | | | |

TABLE 2
MatGAT comparison of sequences SEQ ID NO: 1-14, 59 & 60
(BLOSUM62 alignment matrix)
Light grey shows % identity; dark grey shows % similarity.

| | | 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|---|---|
| 1 | P_islandicum | | 78.3 | 65.6 | 81.7 | 35.9 | 33.9 | 33.9 | 36.7 | 40.0 | 33.3 | 31.7 | 78.3 | 30.6 | 33.3 | 39.7 | 57.1 |
| | (SEQ ID NO: 10) | | | | | | | | | | | | | | | | |
| 2 | P_arsenaticum | 96.6 | | 73.0 | 81.4 | 34.4 | 36.5 | 36.5 | 35.5 | 39.3 | 33.9 | 32.8 | 86.4 | 34.9 | 33.9 | 36.7 | 51.6 |
| | (SEQ ID NO: 11) | | | | | | | | | | | | | | | | |
| 3 | P_calidifontis | 84.1 | 87.3 | | 74.6 | 31.3 | 29.9 | 29.9 | 36.9 | 33.8 | 34.8 | 30.9 | 73.0 | 29.9 | 34.8 | 35.9 | 54.0 |
| | (SEQ ID NO: 13) | | | | | | | | | | | | | | | | |
| 4 | P_aerophilum | 94.9 | 96.6 | 87.3 | | 31.3 | 33.3 | 33.3 | 35.5 | 38.7 | 33.9 | 31.3 | 81.4 | 31.7 | 33.9 | 36.7 | 54.8 |
| | (SEQ ID NO: 12) | | | | | | | | | | | | | | | | |
| 5 | A_permix | 48.4 | 46.9 | 42.2 | 46.9 | | 78.1 | 79.7 | 59.4 | 64.1 | 65.6 | 43.9 | 32.8 | 65.6 | 64.1 | 64.1 | 34.8 |
| | (SEQ ID NO: 7) | | | | | | | | | | | | | | | | |
| 6 | H_buytlicus_0878 | 45.2 | 46.8 | 42.9 | 46.8 | 89.1 | | 98.4 | 66.1 | 67.7 | 74.6 | 48.4 | 34.9 | 76.2 | 73.0 | 74.2 | 37.9 |
| | (SEQ ID NO: 5) | | | | | | | | | | | | | | | | |
| 7 | H_buytlicus_1128 | 45.2 | 46.8 | 42.9 | 46.8 | 89.1 | 100.0 | | 64.5 | 66.1 | 73.0 | 46.9 | 34.9 | 74.6 | 71.4 | 72.6 | 36.4 |
| | (SEQ ID NO: 6) | | | | | | | | | | | | | | | | |
| 8 | M_sedula | 49.2 | 54.2 | 46.0 | 52.5 | 73.4 | 75.8 | 75.8 | | 83.1 | 81.7 | 42.9 | 37.1 | 66.1 | 83.3 | 84.7 | 37.5 |
| | (SEQ ID NO: 3) | | | | | | | | | | | | | | | | |
| 9 | S_acidocaldarius | 49.2 | 52.5 | 44.4 | 52.5 | 78.1 | 79.0 | 79.0 | 89.8 | | 80.0 | 44.4 | 41.0 | 62.9 | 80.0 | 83.1 | 37.5 |
| | (SEQ ID NO: 2) | | | | | | | | | | | | | | | | |
| 10 | S_solfataricus | 41.7 | 50.0 | 42.9 | 50.0 | 75.0 | 82.3 | 82.3 | 88.3 | 86.7 | | 42.2 | 34.4 | 72.6 | 98.3 | 85.0 | 38.5 |
| | (SEQ ID NO: 1) | | | | | | | | | | | | | | | | |
| 11 | I_hospital | 52.5 | 50.8 | 41.3 | 47.5 | 56.3 | 58.1 | 58.1 | 54.2 | 61.0 | 51.7 | | 31.3 | 43.8 | 42.2 | 43.5 | 35.8 |
| | (SEQ ID NO: 9) | | | | | | | | | | | | | | | | |
| 12 | T_neutrophilus | 93.2 | 94.9 | 85.7 | 94.9 | 45.3 | 45.2 | 45.2 | 52.5 | 50.8 | 45.0 | 45.8 | | 30.2 | 33.9 | 38.3 | 54.8 |
| | (SEQ ID NO: 14) | | | | | | | | | | | | | | | | |
| 13 | S_marinus | 41.9 | 41.9 | 42.9 | 41.9 | 79.7 | 87.1 | 87.1 | 79.0 | 80.6 | 85.5 | 53.2 | 41.9 | | 74.2 | 72.6 | 36.4 |
| | (SEQ ID NO: 4) | | | | | | | | | | | | | | | | |
| 14 | S_shibatae | 41.7 | 50.0 | 42.9 | 50.0 | 73.4 | 80.6 | 80.6 | 90.0 | 86.7 | 98.3 | 51.7 | 48.3 | 87.1 | | 85.0 | 38.5 |
| | (SEQ ID NO: 59) | | | | | | | | | | | | | | | | |
| 15 | S_tokodaii | 55.9 | 50.8 | 46.0 | 50.8 | 78.1 | 79.0 | 79.0 | 88.1 | 89.8 | 90.0 | 52.6 | 50.8 | 80.6 | 90.0 | | 43.8 |
| | (SEQ ID NO: 60) | | | | | | | | | | | | | | | | |
| 16 | C_maquilingensis | 73.8 | 73.8 | 71.4 | 75.4 | 50.0 | 46.8 | 46.8 | 45.9 | 50.8 | 49.2 | 54.1 | 73.8 | 45.2 | 49.2 | 52.5 | |
| | (SEQ ID NO: 8) | | | | | | | | | | | | | | | | |

The chimeric protein may comprise one, two or more Cren7 enhancer domains or variants thereof; where there are two or more, each may be the same or different to one or more other Cren7 enhancer domains or variants thereof comprised within the same chimeric protein.

The Sulfolobus solfataricus Cren7 enhancer protein has the amino acid sequence:

(SEQ ID NO: 1)
MSSGKKPVKV KTPAGKEAEL VPEKVWALAP KGRKGVKIGL
FKDPETGKYF RHKLPDDYP I.

As mentioned above, the present inventors have identified that members of a new class of enhancer domain enhance the activity of nucleic acid modifying enzymes. As already described, these domains have since been designated as “Cren7” domains. These Cren7 enhancer domains are structurally and functionally similar to one another, for example to the Sulfolobus solfataricus Cren7 enhancer protein of SEQ ID NO: 1. An illustration of such similarity is set out in FIG. 1B of the publication by Guo et al. (2007, Nucleic Acids Research Advance Access published 20 Dec. 2007, 1-9).

In one aspect of the invention, the Cren7 enhancer domain or variant thereof has DNA binding activity which functions to enhance the activity of the nucleic acid modifying enzyme joined therewith. Without being bound by theory, it is considered that the Cren7 enhancer domains of the invention and variants thereof are non-sequence-specific double stranded DNA binding proteins which function to enhance nucleic acid modifying enzymes in a similar way to Sso7d-like proteins (see above). However, the Cren7 enhancer proteins of the present invention, such as the Sulfolobus solfataricus Cren7 enhancer protein of SEQ ID NO: 1, as well as variants of such proteins, have no significant amino acid similarity or identity to the known Sso7d-like proteins.

A BLASTP analysis against Sso7d or related protein Sac7d does not identify SEQ ID NO:1 and an analysis against SEQ ID NO:1 does not identify Sso7d or Sac7d, indicating that sequence homology is so low as to be undetectable. Table 3 below shows a

MatGAT comparison of Cren7 enhancer domains according to the invention and Sso7d, Sac7d and related protein Ssh7d, demonstrating the low percentage sequence identity of these sequences in comparison with the Cren7 enhancer domains according to the invention. These comparisons were determined using the MatGAT program and the alignment matrix BLOSUM50, with default program parameters used, namely, First Gap 12, Extending Gap 2.

As used herein, the term “chimeric protein” means a protein or polypeptide comprising two or more heterologous domains which are not found in the same relationship to one another in nature.

The domains may be positioned in any arrangement relative to one another. For example, the chimeric protein may comprise a nucleic acid modifying enzyme domain joined at its 3′ end to the 5′ end of an Cren7 enhancer domain or variant thereof, or the nucleic acid modifying enzyme domain may be joined at its 5′ end to the 3′ end of an Cren7 enhancer domain or variant thereof. In another embodiment, the chimeric protein may comprise a nucleic acid modifying enzyme domain joined at its 5′ end to an Cren7 enhancer domain or variant thereof and at its 3′ end to another Cren7 enhancer domain or variant thereof, the Cren7 enhancer domains or variants thereof being the same as or different to one another. The skilled person will appreciate that the chimeric protein may comprise several Cren7 enhancer domains or variants thereof, each of which may the same or different to one or more other Cren7 enhancer domains or variants thereof comprised within the chimeric protein.

The term “joined” means functionally connecting protein domains which have been functionally connected using any method known in the art. By way of non-limiting example, the domains may be recombinantly fused as a single fusion protein, with or without intervening domains or residues, or the domains may be functionally connected by intein-mediated fusion, non-covalent association, and covalent bonding, including disulfide bonding, hydrogen bonding, electrostatic bonding, and conformational bonding such as antibody-antigen or biotin-avidin associations.

TABLE 3
MatGAT comparison of sequences SEQ ID NO: 1-14, 59 & 60 and Sso7d, Sac7d, Ssh7d
(BLOSUM50 alignment matrix)
Light grey shows % identity; dark grey shows % similarity.

| | | 1 | 2 | 3 | 4 | 5 | 6 | 7 | 8 | 9 | 10 | 11 |
|---|---|---|---|---|---|---|---|---|---|---|---|---|
| 1 | P_islandicum (SEQ ID NO: 10) | | 78.3 | 65.6 | 81.7 | 35.9 | 33.9 | 33.9 | 36.7 | 40.0 | 33.3 | 31.7 |
| 2 | P_arsenaticum (SEQ ID NO: 11) | 96.6 | | 73.0 | 81.4 | 34.4 | 36.5 | 36.5 | 35.5 | 39.3 | 33.9 | 32.8 |
| 3 | P_calidifontis (SEQ ID NO: 13) | 84.1 | 87.3 | | 74.6 | 31.3 | 29.9 | 29.9 | 36.9 | 33.8 | 34.8 | 30.9 |
| 4 | P_aerophilum (SEQ ID NO: 12) | 94.9 | 96.6 | 87.3 | | 31.3 | 33.3 | 33.3 | 35.5 | 38.7 | 33.9 | 31.3 |
| 5 | A_permix (SEQ ID NO: 7) | 48.4 | 46.9 | 42.2 | 46.9 | | 78.1 | 79.7 | 59.4 | 64.1 | 65.6 | 43.9 |
| 6 | H_buytlicus_0878 (SEQ ID NO: 5) | 45.2 | 46.8 | 42.9 | 46.8 | 89.1 | | 98.4 | 66.1 | 67.7 | 74.6 | 48.4 |
| 7 | H_buytlicus_1128 (SEQ ID NO: 6) | 45.2 | 46.8 | 42.9 | 46.8 | 89.1 | 100.0 | | 64.5 | 66.1 | 73.0 | 46.9 |
| 8 | M_sedula (SEQ ID NO: 3) | 49.2 | 54.2 | 46.0 | 52.5 | 73.4 | 75.8 | 75.8 | | 83.1 | 81.7 | 42.9 |
| 9 | S_acidocaldarius (SEQ ID NO: 2) | 49.2 | 52.5 | 44.4 | 52.5 | 78.1 | 79.0 | 79.0 | 89.8 | | 80.0 | 44.4 |
| 10 | S_solfataricus (SEQ ID NO: 1) | 41.7 | 50.0 | 42.9 | 50.0 | 75.0 | 82.3 | 82.3 | 88.3 | 86.7 | | 42.2 |
| 11 | I_hospital (SEQ ID NO: 9) | 52.5 | 50.8 | 41.3 | 47.5 | 56.3 | 58.1 | 58.1 | 54.2 | 61.0 | 51.7 | |
| 12 | T_neutrophilus (SEQ ID NO: 14) | 93.2 | 94.9 | 85.7 | 94.9 | 45.3 | 45.2 | 45.2 | 52.5 | 50.8 | 45.0 | 45.8 |
| 13 | S_marinus (SEQ ID NO: 4) | 41.9 | 41.9 | 42.9 | 41.9 | 79.7 | 87.1 | 87.1 | 79.0 | 80.6 | 85.5 | 53.2 |
| 14 | S_shibatae (SEQ ID NO: 59) | 41.7 | 50.0 | 42.9 | 50.0 | 73.4 | 80.6 | 80.6 | 90.0 | 86.7 | 98.3 | 51.7 |
| 15 | S_tokodaii (SEQ ID NO: 60) | 55.9 | 50.8 | 46.0 | 50.8 | 78.1 | 79.0 | 79.0 | 88.1 | 89.8 | 90.0 | 52.6 |
| 16 | C_maquilingensis (SEQ ID NO: 8) | 73.8 | 73.8 | 71.4 | 75.4 | 50.0 | 46.8 | 46.8 | 45.9 | 50.8 | 49.2 | 54.1 |
| 17 | Sso7d | 37.9 | 34.8 | 39.4 | 34.8 | 40.9 | 31.8 | 31.8 | 28.8 | 31.8 | 27.3 | 33.3 |
| 18 | Sac7d | 37.9 | 34.8 | 39.4 | 34.8 | 40.9 | 31.8 | 31.8 | 28.8 | 31.8 | 27.3 | 33.3 |
| 19 | Ssh7d | 37.5 | 34.4 | 35.9 | 34.4 | 42.2 | 37.5 | 37.5 | 35.9 | 39.1 | 34.4 | 32.8 |

| | | 12 | 13 | 14 | 15 | 16 | 17 | 18 | 19 |
|---|---|---|---|---|---|---|---|---|---|
| 1 | P_islandicum (SEQ ID NO: 10) | 78.3 | 30.6 | 33.3 | 39.7 | 57.1 | 13.4 | 13.4 | 10.8 |
| 2 | P_arsenaticum (SEQ ID NO: 11) | 86.4 | 34.9 | 33.9 | 36.7 | 51.6 | 16.4 | 16.4 | 15.6 |
| 3 | P_calidifontis (SEQ ID NO: 13) | 73.0 | 29.9 | 34.8 | 35.9 | 54.0 | 19.1 | 19.1 | 12.3 |
| 4 | P_aerophilum (SEQ ID NO: 12) | 81.4 | 31.7 | 33.9 | 36.7 | 54.8 | 19.4 | 19.4 | 15.6 |
| 5 | A_permix (SEQ ID NO: 7) | 32.8 | 65.6 | 64.1 | 64.1 | 34.8 | 24.7 | 24.7 | 22.7 |
| 6 | H_buytlicus_0878 (SEQ ID NO: 5) | 34.9 | 76.2 | 73.0 | 74.2 | 37.9 | 19.7 | 19.7 | 26.0 |
| 7 | H_buytlicus_1128 (SEQ ID NO: 6) | 34.9 | 74.6 | 71.4 | 72.6 | 36.4 | 19.7 | 19.7 | 26.0 |
| 8 | M_sedula (SEQ ID NO: 3) | 37.1 | 66.1 | 83.3 | 84.7 | 37.5 | 20.3 | 20.3 | 17.6 |
| 9 | S_acidocaldarius (SEQ ID NO: 2) | 41.0 | 62.9 | 80.0 | 83.1 | 37.5 | 15.9 | 15.9 | 14.7 |
| 10 | S_solfataricus (SEQ ID NO: 1) | 34.4 | 72.6 | 98.3 | 85.0 | 38.5 | 14.7 | 14.7 | 14.9 |
| 11 | I_hospital (SEQ ID NO: 9) | 31.3 | 43.8 | 42.2 | 43.5 | 35.8 | 23.9 | 23.9 | 21.5 |
| 12 | T_neutrophilus (SEQ ID NO: 14) | | 30.2 | 33.9 | 38.3 | 54.8 | 16.7 | 16.7 | 15.4 |
| 13 | S_marinus (SEQ ID NO: 4) | 41.9 | | 74.2 | 72.6 | 36.4 | 20.6 | 20.6 | 26.0 |
| 14 | S_shibatae (SEQ ID NO: 59) | 48.3 | 87.1 | | 85.0 | 38.5 | 14.7 | 14.7 | 14.9 |
| 15 | S_tokodaii (SEQ ID NO: 60) | 50.8 | 80.6 | 90.0 | | 43.8 | 18.2 | 18.2 | 20.0 |
| 16 | C_maquilingensis (SEQ ID NO: 8) | 73.8 | 45.2 | 49.2 | 52.5 | | 16.7 | 16.7 | 17.2 |
| 17 | Sso7d | 30.3 | 30.3 | 27.3 | 30.3 | 37.9 | | 100.0 | 79.1 |
| 18 | Sac7d | 30.3 | 30.3 | 27.3 | 30.3 | 37.9 | 100.0 | | 79.1 |
| 19 | Ssh7d | 35.9 | 39.1 | 34.4 | 34.4 | 40.6 | 89.9 | 87.9 | |

The term “domain” as used herein means a unit of a protein or protein complex with a defined function. Thus, the nucleic acid modifying enzyme domain has nucleic acid modifying activity, whilst the Cren7 enhancer domain or variant thereof enhances the activity of the nucleic acid modifying enzyme. Each domain may comprise a polypeptide sequence or subsequence, or a unit having a plurality of polypeptide sequences where the unit has a defined function.

The term “efficiency” as used herein refers to the ability of the nucleic acid modifying enzyme domain to perform its catalytic function under specific reaction conditions. Typically, “efficiency” as defined herein may be indicated by the amount of modified bases generated by the nucleic acid modifying enzyme domain per binding to a nucleic acid.

“Enhanced” in the context of the nucleic acid modifying enzyme domain means improving the activity of the domain by increasing the amount of enzyme product per unit enzyme per unit time.

The Cren7 enhancer domain of the present invention, or variant thereof, may increase the processivity of the nucleic acid modifying enzyme domain, where the enzyme is a processive enzyme.

“Processivity” as used herein means the ability of the nucleic acid modifying enzyme domain to remain attached to its nucleic acid substrate and perform multiple modification reactions. Improved processivity typically means that the nucleic acid nucleic acid modifying enzyme domain can modify relatively longer tracts of nucleic acid.

In one aspect of the invention, the nucleic acid modifying enzyme domain is a functional domain of a processive enzyme that interacts with nucleic acid, such as a nucleic acid polymerase domain, for example a DNA polymerase domain. Alternatively, the nucleic acid modifying enzyme domain may be an RNA polymerase domain with RNA polymerase activity, a reverse transcriptase domain with reverse transcriptase activity, a methylase domain with methylase activity, a 3′ exonuclease domain with 3′ exonuclease activity, a gyrase domain with gyrase activity, a topoisomerase domain with topoisomerase activity, or any functional domain of any other processive enzyme that interacts with nucleic acid.

The nucleic acid modifying enzyme domain may be a ligase domain with ligase activity, an alkaline phosphatase domain with alkaline phosphatise activity, or a nucleic acid kinase domain with nucleic acid kinase activity.

As used herein, a “nucleic acid polymerase” refers to any enzyme that catalyzes polynucleotide synthesis by addition of nucleotide units to a nucleotide chain using a nucleic acid such as DNA as a template. The term includes any variants and recombinant functional derivatives of naturally occurring nucleic acid polymerases, whether derived by genetic modification or chemical modification or other methods known in the art.

The Cren7 enhancer domain or variant thereof may, alternatively or additionally for each property, improve one or more of the following properties of a nucleic acid polymerase domain according to the invention: extension time; salt tolerance; amplification efficiency; and amplification fidelity. For example, the chimeric protein may improve salt tolerance compared with nucleic acid modifying enzyme domain alone by about 20-fold, for example up to 10-fold, or 5-fold. Where the nucleic acid modifying enzyme is a nucleic acid polymerase, the Cren7 enhancer domain or variant thereof may, for example, allow amplification by the nucleic acid polymerase domain at a salt concentration equivalent to up to 200 mM KCl, such as up to 150 mM, 140 mM, 130 mM or up to 100 mM KCl.

Enhancement of the above-mentioned properties of nucleic acid polymerases by the Cren7 enhancer domain or variant thereof provides a substantial improvement and increased application over non-chimeric polymerase. For example, with increased salt tolerance, PCR amplification from low quality of DNA template, such as DNA samples prepared from blood, food and plant sources, becomes more feasible.

The Cren7 enhancer domain of the invention may be one of the group comprising: Sulfolobus solfataricus Cren7 enhancer protein (SEQ ID NO: 1); Sulfolobus acidocaldarius Cren7 enhancer protein (SEQ ID NO: 2); Metallosphaera sedula Cren7 enhancer protein (SEQ ID NO: 3); Staphylothermus marinus Cren7 enhancer protein (SEQ ID NO: 4); Hyperthermus butylicus 0878 Cren7 enhancer protein (SEQ ID NO: 5); Hyperthermus butylicus 1128 Cren7 enhancer protein (SEQ ID NO: 6); Aeropyrum pernix Cren7 enhancer protein (SEQ ID NO: 7); Caldivirga maquilingensis Cren7 enhancer protein (SEQ ID NO: 8); Ignicoccus hospitalis Cren7 enhancer protein (SEQ ID NO: 9); Pyrobaculum islandicum Cren7 enhancer protein (SEQ ID NO: 10); Pyrobaculum arsenaticum Cren7 enhancer protein (SEQ ID NO: 11); Pyrobaculum aerophilum Cren7 enhancer protein (SEQ ID NO: 12); Pyrobaculum calidifontis Cren7 enhancer protein (SEQ ID NO: 13); Thermoproteus neutrophilus Cren7 enhancer protein (SEQ ID NO: 14), Sulfolobus shibatae Cren7 enhancer protein (SEQ ID NO:59); and Sulfolobus tokodaii and Cren7 enhancer protein (SEQ ID NO:60). The variant of a Cren7 enhancer domain may be a functional or structural variant having at least 35% sequence identity (for example, at least 40%, 45%, 50%, 55%, 60%, 65%, 70%, 75%, 80%, 85%, 90%, 95%, 96%, 97%, 98% or even 99% sequence identity) with any of the Cren7 enhancer proteins of SEQ ID NO: 1-14, 59 or 60, determined using BLASTP with any of SEQ ID NO:1-14, 59 or 60, as appropriate, used as the base sequence.

In Hyperthermus butylicus it has been found that, unusually, two Cren7 proteins are expressed. The numbers used to differentiate the proteins (0878 and 1128) are the ORF (Open reading frame) numbers, as given in the genome, for each of the two copies.

The Sulfolobus acidocaldarius Cren7 enhancer protein has the amino acid sequence:

(SEQ ID NO: 2)
MSEKKRVRVR TPGGKELELT PEKTWVLAPK GRKGVKIGLF
KDPESGKYFR HKLPDDYPV.

The Metallosphaera sedula Cren7 enhancer protein has the amino acid sequence:

(SEQ ID NO: 3)
MTYKKAVKIK TPGGKEAELA PEKAWTLAPK GRKGVKIGLF
KDPESGKYFR HKLPDDYPV.

The Staphylothermus marinus Cren7 enhancer protein has the amino acid sequence:

(SEQ ID NO: 4)
MAACKDAVKV KTLSGKEVEL VPKKVWQLSP KGRKGVKVG
LFQDPETGKY FAKVPDDYP ICG.

The Hyperthermus butylicus 0878 Cren7 enhancer protein has the amino acid sequence:

(SEQ ID NO: 5)
MACEKPVKVR DPTTGKEVEL VPIKVWQLAP KGRKGVKIG
LFKSPETGKY FAKVPDDYP ICS.

The Hyperthermus butylicus 1128 Cren7 enhancer protein has the amino acid sequence:

(SEQ ID NO: 6)
MACEKPVKVR DPTTGKEVEL VPIKVWQLAP RGRKGVKIG
LFKSPETGKY FRAKVPDDYP ICS.

The Aeropyrum pernix Cren7 enhancer protein has the amino acid sequence:

(SEQ ID NO: 7)
MSQKQLPPVK VRDPTTGKEV ELTPIKVWKL SPRGRRGVKI
GLFKSPETGK FRAKVPDDY PETG.

The Caldivirga maquilingensis Cren7 enhancer protein has the amino acid sequence:

(SEQ ID NO: 8)
MLFMFISHYA VYLLTGMAVN VQQYLNKEYE VECDGQMVRL
KPVKAWVLQP GRKGVVIGL FKCPNGKTLR KAIGKIE.

The Ignicoccus hospitalis Cren7 enhancer protein has the amino acid sequence:

(SEQ ID NO: 9)
MPKCPKCGAE VKEPIKTWVL APKGRKGVII GLFRCPNGHY
FRAKVGEAPP KKEAA.

The Pyrobaculum islandicum Cren7 enhancer protein has the amino acid sequence:

(SEQ ID NO: 10)
MEEVLDREYE VEYGGRKYRL KPVKAWVLQP PGKPGVVIAL
FKLPDGKTIR KVIMKLPPS.

The Pyrobaculum arsenaticum Cren7 enhancer protein has the amino acid sequence:

(SEQ ID NO: 11)
MAEEILNREY EVEYGGKRYI LRPIKAWVLQ PPGKPGVVVA
LFRLPDGKTV RKVVMKLPP.

The Pyrobaculum aerophilum Cren7 enhancer protein has the amino acid sequence:

(SEQ ID NO: 12)
MAEEILNREY EVEYEGRKYF LRPVKAWVLQ PPGKPGVVVA
LFKLPNGKSI RKVIMRLPP.

The Pyrobaculum calidifontis Cren7 enhancer protein has the amino acid sequence:

(SEQ ID NO: 13)
MDQDVAEEIL NKEYEVVYEG KRFLLKPAKA WVLQPPGKPG
VIVALFKLPN GKTVRKVIAR LPP.

The Thermproteus neutrophilus Cren7 enhancer protein has the amino acid sequence:

(SEQ ID NO: 14)
MAEEILNREY EVEYGGKRYW LRPSKAWVLQ PPGKPGVVIA
LFKLPDGRTV RKAIMRLPP.

The Sulfolobus shibatae Cren7 enhance protein has the amino acid sequence:

(SEQ ID NO: 59)
MSSGKKAVKV KTPAGKEAEL VPEKVWALAP KGRKGVKIGL
FKDPETGKYF RHKLPDDYPI.

The Sulfolobus tokodaii Cren7 enhance protein has the amino acid sequence:

(SEQ ID NO: 60)
MAEKKVKVKT PSGKEAELAP EKVWVLAPKG RKGVKIGLFK
DPETGKYFRH KLPDDYP

The majority of the Cren7 enhancer proteins for use in the invention are deemed to be “hypothetical proteins” in the prior art. However, the present application demonstrates that these proteins form a class of proteins with putative DNA binding activity that enhances nucleic acid modifying activity (such as DNA polymerase activity). This is now confirmed by the adoption of the Cren7 domain classification as standard in the art for use with databases of protein sequences, as discussed above.

The Cren7 enhancer domain, or variant thereof, for use in the present invention may comprise the conserved amino acid sequence:

G-X₁-X₂-X₁-X₁-X₃-X₁-P-X₁-K-X₄-W-X₁-L-X₁-P-X₂-G-X₅-X₁-G-V-X₁-X₆-X₇-L-F-X₈-X₁-P-X₉-X₁₀-G-X₁₁-X₁-X₁₂-R-X₁-X₁-X₁₃ (SEQ ID NO: 15). Alternatively, the Cren7 enhancer domain, or variant thereof, of the present invention may comprise the conserved amino acid sequence:

X₂-X₁-X1-X1-X₁₀-G-X₁-X₁-X₁-X₁-X₃-X₁-P-X₁-K-X₄-W-X₁-L-X₁-P-X₁-G-X₅-X₁-G-V-X₁-X₆-X₇-L-F-X₈-X₁-P-X₉-X₁₀-G-X₁₁-X₁-X₁₂-R-X₁-X₁-X₁₃ (SEQ ID NO:61).

In either case, independently,

X₁ is any amino acid,

X₂ is K, R or E,

X₃ is L or no amino acid,

X₄ is A, V or T,

X₅ is K or R,

X₆ is I or V,

X₇ is G or A,

X₈ is K, R or Q,

X₉ is D, N or E,

X₁₀ is any or no amino acid,

X₁₁ is K or H,

X₁₂ is I, V or F, and

X₁₃ is I, V or L.

In one embodiment, the Cren7 enhancer domain comprises or consists essentially of one of the group comprising: SEQ ID NO:1, SEQ ID NO: 6, SEQ ID NO: 7, The variant of the Cren7 enhancer domain may comprise or consist essentially of a functional or structural variant having at least 40% sequence identity with any of the Cren7 enhancer proteins SEQ ID NO:1, SEQ ID NO:6, SEQ ID NO:7.

The Cren7 enhancer domain or variant thereof of the present invention may have DNA binding activity.

The nucleic acid polymerase domain of the invention may comprise or consist essentially of a thermostable DNA polymerase or a functional mutant, variant or derivative thereof.

As used herein, “thermostable” DNA polymerase means a DNA polymerase which is relatively stable to heat and functions at high temperatures, for example 45-100° C., as compared, for example, to a non-thermostable form of DNA polymerase. For example, a thermostable DNA polymerase derived from thermophilic organisms such as Pyrococcus furiosus (Pfu; see for example Lundberg et al., 1991, Gene, 108: 1-6), Methanococcus jannaschii, Archaeoglobus fulgidus or P. horikoshii are more stable and active at elevated temperatures as compared to a DNA polymerase from E. coli. Representative thermostable polymerases include Pfu as well as polymerases extracted from the thermophilic bacteria Thermus flavus, Thermus aquaticus, Thermus brockianus, Thermus ruber, Thermus thermophilus, Bacillus stearothermophilus, Thermus lacteus, Thermus rubens, Thermotoga maritima, or from thermophilic archaea Thermococcus litoralis, and Methanothermus fervidus. Thermostable DNA polymerases for use in the present invention include Taq, KlenTaq, Tne, Tma, Pfu, Tfl, Tth, Stoffel fragment, VENT™ DNA polymerase, DEEPVENT™ DNA polymerase, KOD, Tgo, JDF3 and the like (see for example U.S. Pat. No. 5,436,149; U.S. Pat. No. 4,889,818; U.S. Pat. No. 4,965,188; U.S. Pat. No. 5,079,352; U.S. Pat. No. 5,614,365; U.S. Pat. No. 5,616,494; U.S. Pat. No. 5,374,553; U.S. Pat. No. 5,512,462; WO92/06188; WO92/06200; WO96/10640; Engelke et al., 1990, Anal. Biochem. 191: 396-400; Lawyer et al., 1993, PCR Meth. Appl. 2: 275-287; Flaman et al., 1994, Nuc. Acids Res. 22: 3259-3260).

Specific, non-limiting examples of chimeric proteins according to the invention which include an Cren7 enhancer domain and a thermostable DNA polymerase domain are:

(1) a chimeric protein in which the Cren7 enhancer domain from Aeropyrum pernix (“Ape”) is joined with KlenTaq, Taq or Pfu thermostable DNA polymerase (such as the fusion proteins of SEQ ID NOs: 16, 17 and 18, respectively, shown in the experimental section below);

(2) a chimeric protein in which the Cren7 enhancer domain from Sulfolobus solfataricus (“Sso”) is joined with KlenTaq, Taq or Pfu thermostable DNA polymerase (such as the fusion proteins of SEQ ID NOs: 19, 20 and 21, respectively, shown in the experimental section below);

(3) a chimeric protein in which the Cren7 enhancer domain from Pyrobaculum islandicum (“Pis”) is joined with KlenTaq (such as the fusion protein of SEQ ID NO: 22 shown in the experimental section below), Taq or Pfu; and

(4) a chimeric protein in which the Cren7 enhancer domain from Hyperthermus butylicus (“Hbu”) joined with Taq or Pfu thermostable DNA polymerase (such as the fusion proteins of SEQ ID NOs: 23 and 24 respectively, shown in the experimental section below) or KlenTaq.

Alternatively, the nucleic acid polymerase domain of the invention may comprise or consist essentially of a mesophilic DNA polymerase or a functional mutant, variant or derivative thereof.

The mesophilic DNA polymerase may, for example, be T7 DNA polymerase, T5 DNA polymerase, T4 DNA polymerase, Klenow fragment DNA polymerase or DNA polymerase III (see for example U.S. Pat. No. 5,270,179; U.S. Pat. No. 5,047,342; Barnes, 1992, Gene 112:29-35).

Alternatively, the nucleic acid polymerase domain of the invention may comprise or consist essentially of intermediate temperature DNA polymerases, namely, those which work optimally within the range 60-70° C., for example, at or around about 65° C. Examples are the polymerases obtainable from Bacillus or Geobacillus species.

The present invention encompasses variants of the Cren7 enhancer domains and nucleic acid modifying enzyme (for example, polymerase) domains. As used herein, a “variant” means a polypeptide in which the amino acid sequence differs from the base sequence from which it is derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type.

By “conservative substitution” is meant the substitution of an amino acid by another amino acid of the same class, in which the classes are defined as follows:

| Class | Amino acid examples |
|---|---|
| Nonpolar: | A, V, L, I, P, M, F, W |
| Uncharged polar: | G, S, T, C, Y, N, Q |
| Acidic: | D, E |
| Basic: | K, R, H. |

As is well known to those skilled in the art, altering the primary structure of a peptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptide's conformation.

Non-conservative substitutions are possible provided that these do not interrupt or interfere with the function of the DNA binding domain polypeptides.

Broadly speaking, fewer non-conservative substitutions will be possible without altering the biological activity of the polypeptides. Suitably, variants may be structural variants having at least 35% identical, 40% identical, 45% identical, 50% identical, 55% identical, 60% identical, 65% identical, for example at least 70% or 75% identical, such as at least 80%, 85%, 90%, 95%, 96%, 97%, 98% or even 99% identical to the base sequence.

Functional mutants, variants or derivatives of the above-mentioned chimeric proteins are also covered by the present invention. Such mutants, variants or derivatives are considered to be “functional” if they retain the same or similar properties and/or activity to the chimeric proteins of the invention as described herein. For example, where a chimeric protein is a DNA polymerase having a given activity, a variant is considered functional where the level of activity is at least 60%, preferably at least 70%, more preferably at least 80%, yet more preferably 90%, 95%, 96%, 97%, 98%, 99% or 100% that of the chimeric protein. The given activity may be determined by any standard measure, for example (in the case of a DNA polymerase), the number of bases of nucleotides of the template sequence which can be replicated in a given time period. The skilled person is routinely able to determine such properties and activities and examples of such methods are provided in the current specification. Functional mutants of the nucleic acid polymerases may include mutants which have been modified so as to remove other properties or activities not of interest, such as exonuclease activity.

Also provided according to the present invention is a composition comprising the chimeric protein as defined herein. Preferably the composition further comprises at least one component necessary or beneficial to activity of the nucleic acid modifying enzyme domain of the chimeric protein and/or the Cren7 enhancer domain. For example, where the nucleic acid modifying enzyme domain is a DNA polymerase, the composition may comprise a buffer and/or other reagents required to enable a polymerase chain reaction to be carried out.

According to a further aspect of the invention there is provided an isolated nucleic acid encoding the chimeric protein as defined herein.

For example, the nucleic acid may have the sequence of any one of SEQ ID NOs: 25-33 (which encode the chimeric protein of SEQ ID NOs: 16-24, respectively).

Using the standard genetic code, a nucleic acid encoding the polypeptides may readily be conceived and manufactured by the skilled person. The nucleic acid may be DNA or RNA, and where it is a DNA molecule, it may for example comprise a cDNA or genomic DNA.

The invention encompasses variant nucleic acids encoding the chimeric protein. The term “variant” in relation to a nucleic acid sequences means any substitution of, variation of, modification of, replacement of deletion of, or addition of one or more nucleic acid(s) from or to a polynucleotide sequence, providing the resultant polypeptide sequence encoded by the polynucleotide exhibits at least the same properties as the polypeptide encoded by the basic sequence. The term therefore includes allelic variants and also includes a polynucleotide which substantially hybridises to the polynucleotide sequence of the present invention. Such hybridisation may occur at or between low and high stringency conditions. In general terms, low stringency conditions can be defined as hybridisation in which the washing step takes place in a 0.330-0.825 M NaCl buffer solution at a temperature of about 40-48° C. below the calculated or actual melting temperature (Tₘ) of the probe sequence (for example, about ambient laboratory temperature to about 55° C.), while high stringency conditions involve a wash in a 0.0165-0.0330 M NaCl buffer solution at a temperature of about 5-10° C. below the calculated or actual Tₘ of the probe (for example, about 65° C.). The buffer solution may, for example, be SSC buffer (0.15M NaCl and 0.015M tri-sodium citrate), with the low stringency wash taking place in 3×SSC buffer and the high stringency wash taking place in 0.1×SSC buffer. Steps involved in hybridisation of nucleic acid sequences have been described for example in Sambrook et al. (1989; Molecular Cloning, Cold Spring Harbor Laboratory Press, Cold Spring Harbor).

Typically, variants have 60% or more of the nucleotides in common with the nucleic acid sequence of the present invention, more typically 65%, 70%, 80%, 85%, or even 90%, 95%, 98% or 99% or greater sequence identity. The percentage sequence identity of nucleic acid sequences may be determined using, for example, the BLASTN software, available at http://blast.ncbinlm.nih.gov/Blast.cgi (accessible on 6 Jan. 2009).

Variant nucleic acids of the invention may be codon-optimised for expression in a particular host cell.

Chimeric proteins and nucleic acids of the invention may be prepared synthetically using conventional synthesisers. Alternatively, they may be produced using recombinant DNA technology or isolated from natural sources followed by any chemical modification, if required. In these cases, a nucleic acid encoding the chimeric protein is incorporated into a suitable expression vector, which is then used to transform a suitable host cell, such as a prokaryotic cell such as E. coli. The transformed host cells are cultured and the protein isolated therefrom. Vectors, cells and methods of this type form further aspects of the present invention.

Sequence identity between nucleotide and amino acid sequences can be determined by comparing an alignment of the sequences. When an equivalent position in the compared sequences is occupied by the same amino acid or base, then the molecules are identical at that position. Scoring an alignment as a percentage of identity is a function of the number of identical amino acids or bases at positions shared by the compared sequences. When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. In addition to the BLASTP, BLASTN and MatGAT programs discussed above, further examples include the Gap program (Needleman & Wunsch, 1970, J. Mol. Biol. 48: 443-453) and the FASTA program (Altschul et al., 1990, J. Mol. Biol. 215: 403-410). Gap and FASTA are available as part of the Accelrys GCG Package Version 11.1 (Accelrys, Cambridge, UK), formerly known as the GCG Wisconsin Package. The PASTA program can alternatively be accessed publicly from the European Bioinformatics Institute (http://www.ebi.ac.uk/fasta as accessed on 6 Jan. 2009) and the University of Virginia (http://fasta.biotech.virginia.edu/fasts_www/cgi) or (http://fasta.bioch.virginia.edu/fasta_www2/fasta_list2.shtml as accessed on 6 Jan. 2009). FASTA may be used to search a sequence database with a given sequence or to compare two given sequences (see http://fasta.bioch.virginia.edu/fasta_www/cgi/search_frm2.cgi as accessed on 6 Jan. 2009). Typically, default parameters set by the computer programs should be used when comparing sequences. The default parameters may change depending on the type and length of sequences being compared. A sequence comparison using the FASTA program may use default parameters of Ktup=2, Scoring matrix=Blosum50, gap=−10 and ext=−2.

The invention also provides a vector comprising the isolated nucleic acid as defined herein and a host cell transformed with such a vector, or transformed with the isolated nucleic acid as defined herein.

The invention also provides a kit comprising the chimeric protein as defined herein, and/or the composition as defined herein, and/or the isolated nucleic acid as defined herein, and/or the vector as defined herein, and/or a host cell transformed as described herein, together with packaging materials therefor.

In a further aspect there is provided a method of modifying a nucleic acid, comprising:

(1) contacting the nucleic acid with the chimeric protein as defined herein under conditions which allow activity of the nucleic acid modifying enzyme (such as nucleic acid polymerase) domain; and

(2) permitting the nucleic acid modifying enzyme domain to modify the nucleic acid.

A further method according to the invention is one of catalysing the synthesis of a polynucleotide from a target nucleic acid, comprising the steps of:

(1) providing a chimeric protein comprising a nucleic acid polymerase as defined herein; and

(2) contacting the target nucleic acid with the chimeric protein under conditions which allow the addition by the chimeric protein of nucleotide units to a nucleotide chain using the target nucleic acid, thereby synthesising the polynucleotide.

In another aspect of the invention there is provided a method of amplifying a sequence of a target nucleic acid using a thermocycling reaction, comprising the steps of:

(1) contacting the target nucleic acid with a chimeric protein comprising a nucleic acid polymerase as defined herein; and

(2) incubating the target nucleic acid with the chimeric protein under thermocycling reaction conditions which allow amplification of the target nucleic acid.

### BRIEF DESCRIPTION OF FIGURES

Particular non-limiting embodiments of the present invention will now be described with reference to the following figures, in which:

FIG. 1 shows a sodium dodecyl sulphate (SDS) polyacrylamide gel electrophoresis (PAGE) gel in which KlenTaq and various KlenTaq fusion proteins have been separated;

FIG. 2 shows an SDS PAGE gel in which Taq and various Taq fusion proteins have been separated;

FIG. 3 shows an SDS PAGE gel in which Pfu and various Pfu fusion proteins have been separated;

FIG. 4 shows an agarose gel of PCR reaction samples evidencing the amplification efficiency of the Sso enhancer domain-KlenTaq fusion protein (“Sso-ktaq-enhancer”) compared with KlenTaq DNA polymerase (“Ktaq”) in the presence of differently sized DNA templates as indicated;

FIG. 5 shows an agarose gel of PCR reaction samples evidencing the amplification efficiency of the Hbu Cren7 enhancer domain-Pfu fusion protein (“Hbu-Pfu-enhancer”) compared with Pfu DNA polymerase (“Pfu”) in the presence of differently sized DNA templates as indicated;

FIG. 6 shows an agarose gel of PCR reaction samples evidencing the amplification efficiency of Pfu DNA polymerase in the presence of increasing amounts of KCl as shown in mM;

FIG. 7 shows an agarose gel of PCR reaction samples evidencing the amplification efficiency of Ape Cren7 enhancer domain-Pfu fusion protein in the presence of increasing amounts of KCl as shown in mM, as compared with the results of FIG. 6;

FIG. 8 shows an agarose gel of PCR reaction samples evidencing the amplification efficiency of KlenTaq DNA polymerase in the presence of increasing amounts of KCl as shown in mM;

FIG. 9 shows an agarose gel of PCR reaction samples evidencing the amplification efficiency of Ape Cren7 enhancer domain-KlenTaq fusion protein in the presence of increasing amounts of KCl as shown in mM, as compared with the results of FIG. 8;

FIG. 10 shows qPCR results using Taq polymerase (A) and Ape Cren7 enhancer domain-Taq fusion polymerase (B); and

FIG. 11 shows qPCR results using Taq polymerase (A), Ape Cren 7 enhancer domain-Taq fusion polymerase (B) and Sso Cren7 enhancer domain-Taq fusion polymerase (C).

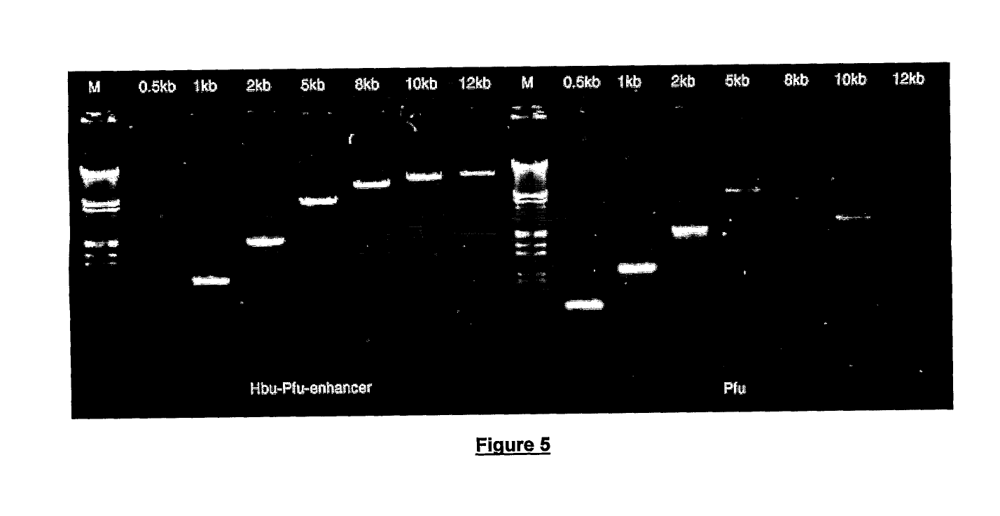

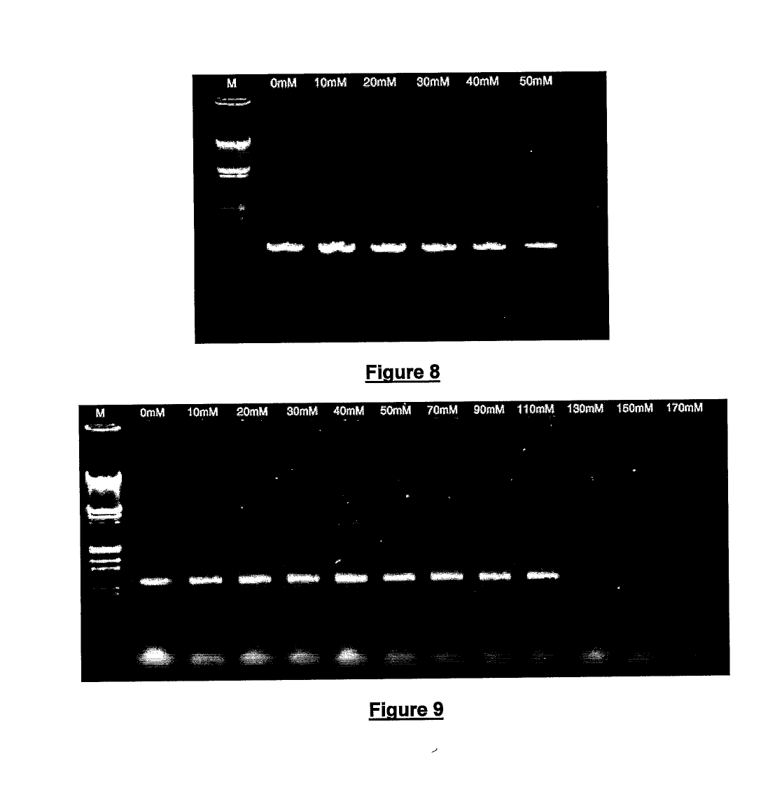

In FIGS. 1-3, lanes marked “M” represent molecular weight markers, with values given in kDa. In FIGS. 4-9, lanes marked “M” are DNA molecular weight markers.

### EXAMPLES

1. Construction of Cren7 Enhancer Domain-KlenTaq DNA Polymerase Fusion Proteins

The entire Aeropyrum pernix (“Ape”) Cren7 enhancer domain gene was amplified by PCR from genomic DNA using the following primer set:

Upper
(SEQ ID NO: 34)
5′-GATATCCATATGAGCCAGAAGCAACTACCA-3′
Lower
(SEQ ID NO: 35)
5′-GAATTCCATATGGGTACCCCCGGTCTCGGGGTAGTCGT-3′.

The forward and reverse primers contained an NdeI site. The reverse primer in addition coded for a small linker peptide (sequence Gly-Thr-His) between the end of the Cren7 enhancer domain gene and the ATG of the NdeI site.

The PCR reactions contained Pfu DNA polymerase reaction buffer (20 mM Tris-HCl (pH 8.8), 2 mM MgSO₄, 10mM KCl, 10 mM (NH₄)₂SO₄, 1% Triton® X-100), 200 μM each dNTP, 0.5μM forward and reverse primers, 100 ng genomic DNA and 0.05 u/μl of a mixture of Taq and Pfu (20:1 ratio) DNA polymerases.

The cycling protocol was 94° C. for 60s; 25 cycles of 94° C. for 10 s and 72° C. for 15 s.

The entire Sulfolobus solfataricus (“Sso”) Cren7 enhancer domain gene was similarly amplified by PCR from genomic DNA with the following primer set:

Upper
(SEQ ID NO: 36)
5′-GATATCCATATGAGTTCGGGTAAAAAACC-3′
Lower
(SEQ ID NO: 37)
5′-GAATTCCATATGGGTACCTATTGGATAATCATCTGGTA-3′.

The entire Pyrobaculum islandicum (“Pis”) Cren7 enhancer domain gene was similarly amplified by PCR from genomic DNA with the following primer set:

Upper
(SEQ ID NO: 38)
5′-GATATCCATATGGAAGAGGTCTTAGATCGT-3′
Lower
(SEQ ID NO: 39)
5′-GAATTCCATATGGGTAACGCTAGGCGGCAATTTCATTA-3′.

An expression vector pTTQ18KTAQ was obtained by cloning KlenTaq (see U.S. Pat. No. 5,616,494 and Barnes, 1992, cited above) into the vector pTTQ18 (Stark, 1987, Gene 51: 255-267) following codon-optimisation of the DNA polymerase using standard techniques for expression in E. coli cells. The amplified Pis Cren7 enhancer domain genes were digested with NdeI and cloned into pTTQ18KTAQ. Ligated DNA was used to transform E. coli cells TOP10F′ (Invitrogen) and transformants plated on a Kanamycin plate. In plasmid mini-prep screening, approximately one out of ten was found to contain the correct size and orientated insert. E. coli cells carrying a sequenced insert plasmid were induced by addition of IPTG for 4 hours. Cells were lysed by sonication. The clarified lysate was then heat treated at 70° C. for 30 min to inactivate the endogenous polymerases.

Cren7 enhancer domain-KlenTaq DNA polymerase fusions (“KlenTaq fusions”) were subjected to electrophoresis in an 8% SDS PAGE gel. A major band of about 70 kDa was detected as compared to 62 kDa for similarly induced KlenTaq DNA polymerase without Cren7 enhancer domain, as shown in FIG. 1. This correlates with the predicted molecular weights of around 70 kDa for the chimeric protein and around 62 kDa for the non-chimeric KlenTaq DNA polymerase.

The Ape Cren7 enhancer domain-KlenTaq fusion protein cloned as described above has following DNA sequence (5′-3′):

(SEQ ID NO: 25)
atgagccagaagcaactaccacctgtgaaggtcagggacccgactaca
ggcaaggaggtcgagctaacgccaatcaaagtgtggaagctatcgccg
agggggaggaggggcgtcaagataggtctcttcaagagccccgagacg
ggcaagtacttcagggccaaggtgcccgacgactaccccgagaccggg
ggtacccatatgggtctgctgcacgaattcggtctgctggaatctccg
aaagcgctggaagaagcgccgtggccgccgccggaaggtgcgttcgtt
ggtttcgttctgtctcgtaaagaaccgatgtgggcggacctgctggcg
ctggcggcggcgcgtggtggtcgtgttcaccgtgcgccggaaccttat
aaagccctcagggacctgaaggaggcgcgggggcttctcgccaaagac
ctgagcgttctggccctgagggaaggccttggcctcccgcccggcgac
gaccccatgctcctcgcctacctcctggacccttccaacaccaccccc
gagggggtggcccggcgctacggcggggagtggacggaggaggcgggg
gagcgggccgccctttccgagaggctcttcgccaacctgtgggggagg
cttgagggggaggagaggctcctttggctttaccgggaggtggagagg
cccctttccgctgtcctggcccacatggaggccacgggggtgcgcctg
gacgtggcctatctcagggccttgtccctggaggtggccgaggagatc
gcccgcctcgaggccgaggtcttccgcctggccggccaccccttcaac
ctcaactcccgggaccagctggaaagggtcctctttgacgagctaggg
cttcccgccatcggcaagacggagaagaccggcaagcgctccaccagc
gccgccgtcctggaggccctccgcgaggcccaccccatcgtggagaag
atcctgcagtaccgggagctcaccaagctgaagagcacctacattgac
cccttgccggacctcatccaccccaggacgggccgcctccacacccgc
ttcaaccagacggccacggccacgggcaggctaagtagctccgatccc
aacctccagaacatccccgtccgcaccccgcttgggcagaggatccgc
cgggccttcatcgccgaggaggggtggctattggtggccctggactat
agccagatagagctcagggtgctggcccacctctccggcgacgagaac
ctgatccgggtcttccaggaggggcgggacatccacacggagaccgcc
agctggatgttcggcgtcccccgggaggccgtggaccccctgatgcgc
cgggcggccaagaccatcaacttcggggtcctctacggcatgtcggcc
caccgcctctcccaggagctagccatcccttacgaggaggcccaggcc
ttcattgagcgctactttcagagcttccccaaggtgcgggcctggatt
gagaagaccctggaggagggcaggaggcgggggtacgtggagaccctc
ttcggccgccgccgctacgtgccagacctagaggcccgggtgaagagc
gtgcgggaggcggccgagcgcatggccttcaacatgcccgtccagggc
accgccgccgacctcatgaagctggctatggtgaagctcttccccagg
ctggaggaaatgggggccaggatgctccttcaggtccacgacgagctg
gtcctcgaggccccaaaagagagggcggaggccgtggcccggctggcc
aaggaggtcatggagggggtgtatccctggccgtgcccctggaggtgg
aggtggggataggggaggactggctctccgccaaggagtga,

and a corresponding amino acid sequence:

(SEQ ID NO: 16)
MSQKQLPPVKVRDPTTGKEVELTPIKVWKLSPRGRRGVKIGLFKSPET
GKYFRAKVPDDYPETGGTHMGLLHEFGLLESPKALEEAPWPPPEGAFV
GFVLSRKEPMWADLLALAAARGGRVHRAPEPYKALRDLKEARGLLAKD
LSVLALREGLGLPPGDDPMLLAYLLDPSNTTPEGVARRYGGEWTEEAG
ERAALSERLFANLWGRLEGEERLLWLYREVERPLSAVLAHMEATGVRL
DVAYLRALSLEVAEEIARLEAEVFRLAGHPFNLNSRDQLERVLFDELG
LPAIGKTEKTGKRSTSAAVLEALREAHPIVEKILQYRELTKLKSTYID
PLPDLIHPRTGRLHTRFNQTATATGRLSSSDPNLQNIPVRTPLGQRIR
RAFIAEEGWLLVALDYSQIERLRVLAHLSGDENLIRVFQEGRDIHTET
ASWMFGVPREAVDPLMRRAAKTINFGVLYGMSAHRLSQELAIPYEEAQ
AFIERYFQSFPKVRAWIEKTLEEGRRRGYVETLFGRRRYVPDLEARVK
SVREAAERMAFNMPVQGTAADLMKLAMVKLFPRLEEMGARMLLQVHDE
LVLEAPKERAEAVARLAKEVMEGVYPLAVPLEVEVGIGEDWLSAKE.

The Sso Cren7 enhancer domain-KlenTaq fusion protein cloned as described above has the following DNA sequence (5′-3′):

(SEQ ID NO: 28)
atgagttcgggtaaaaaaccagtaaaagtaaaaacaccagctggtaaa
gaggctgaattggttccagaaaaagtatgggcattagcaccaaagggt
agaaaaggtgtaaagataggtttatttaaagatccagaaactgggaaa
tacttcagacataagctaccagatgattatccaataggtacccatatg
ggtctgctgcacgaattcggtctgctggaatctccgaaagcgctggaa
gaagcgccgtggccgccgccggaaggtgcgttcgttggtttcgttctg
tctcgtaaagaaccgatgtgggcggacctgctggcgctggcggcggcg
cgtggtggtcgtgttcaccgtgcgccggaaccttataaagccctcagg
gacctgaaggaggcgcgggggcttctcgccaaagacctgagcgttctg
gccctgagggaaggccttggcctcccgcccggcgacgaccccatgctc
ctcgcctacctcctggacccttccaacaccacccccgagggggtggcc
cggcgctacggcggggagtggacggaggaggcgggggagcgggccgcc
ctttccgagaggctcttcgccaacctgtgggggaggcttgagggggag
gagaggctcctttggctttaccgggaggtggagaggcccctttccgct
gtcctggcccacatggaggccacgggggtgcgcctggacgtggcctat
ctcagggccttgtccctggaggtggccgaggagatcgcccgcctcgag
gccgaggtcttccgcctggccggccaccccttcaacctcaactcccgg
gaccagctggaaagggtcctctttgacgagctagggcttcccgccatc
ggcaagacggagaagaccggcaagcgctccaccagcgccgccgtcctg
gaggccctccgcgaggcccaccccatcgtggagaagatcctgcagtac
cgggagctcaccaagctgaagagcacctacattgaccccttgccggac
ctcatccaccccaggacgggccgcctccacacccgcttcaaccagacg
gccacggccacgggcaggctaagtagctccgatcccaacctccagaac
atccccgtccgcaccccgcttgggcagaggatccgccgggccttcatc
gccgaggaggggtggctattggtggccctggactatagccagatagag
ctcagggtgctggcccacctctccggcgacgagaacctgatccgggtc
ttccaggaggggcgggacatccacacggagaccgccagctggatgttc
ggcgtcccccgggaggccgtggaccccctgatgcgccgggcggccaag
accatcaacttcggggtcctctacggcatgtcggcccaccgcctctcc
caggagctagccatcccttacgaggaggcccaggccttcattgagcgc
tactttcagagcttccccaaggtgcgggcctggattgagaagaccctg
gaggagggcaggaggcgggggtacgtggagaccctcttcggccgccgc
cgctacgtgccagacctagaggcccgggtgaagagcgtgcgggaggcg
gccgagcgcatggccttcaacatgcccgtccagggcaccgccgccgac
ctcatgaagctggctatggtgaagctcttccccaggctggaggaaatg
ggggccaggatgctccttcaggtccacgacgagctggtcctcgaggcc
ccaaaagagagggcggaggccgtggcccggctggccaaggaggtcatg
gagggggtgtatcccctggccgtgcccctggaggtggaggtggggata
ggggaggactggctctccgccaaggagtga,

and a corresponding amino acid sequence:

(SEQ ID NO: 19)
MSSGKKPVKVKTPAGKEAELVPEKVWALAPKGRKGVKIGLFKDPETGK
YFRHKLPDDYPIGTHMGLLHEFGLLESPKALEEAPWPPPEGAFVGFVL
SRKEPMWADLLALAAARGGRVHRAPEPYKALRDLKEARGLLAKDLSVL
ALREGLGLPPGDDPMLLAYLLDPSNTTPEGVARRYGGEWTEEAGERAA
LSERLFANLWGRLEGEERLLWLYREVERPLSAVLAHMEATGVRLDVAY
LRALSLEVAEEIARLEAEVFRLAGHPFNLNSRDQLERVLFDELGLPAI
GKTEKTGKRSTSAAVLEALREAHPIVEKILQYRELTKLKSTYIDPLPD
LIHPRTGRLHTRFNQTATATGRLSSSDPNLQNIPVRTPLGQRIRRAFI
AEEGWLLVALDYSQIELRVLAHLSGDENLIRVFQEGRDIHTETASWMF
GVPREAVDPLMRRAAKTINFGVLYGMSAHRLSQELAIPYEEAQAFIER
YFQSFPKVRAWIEKTLEEGRRRGYVETLFGRRRYVPDLEARVKSVREA
AERMAFNMPVQGTAADLMKLAMVKLFPRLEEMGARMLLQVHDELVLEA
PKERAEAVARLAKEVMEGVYPLAVPLEVEVGIGEDWLSAKE.

The Pis Cren7 enhancer domain-KlenTaq fusion protein cloned as described above has the following DNA sequence (5′-3′):

(SEQ ID NO: 31)
atggaagaggtcttagatcgtgaatacgaagtggaatacggcgggaga
aaataccggctaaagccagttaaagcatgggttctccagccccctggc
aaaccaggtgtcgtcatagccctctttaaactaccagatggaaaaact
attaggaaggtgataatgaaattgccgcctagcgttacccatatgggt
ctgctgcacgaattcggtctgctggaatctccgaaagcgctggaagaa
gcgccgtggccgccgccggaaggtgcgttcgttggtttcgttctgtct
cgtaaagaaccgatgtgggcggacctgctggcgctggcggcggcgcgt
ggtggtcgtgttcaccgtgcgccggaaccttataaagccctcagggac
ctgaaggaggcgcgggggcttctcgccaaagacctgagcgttctggcc
ctgagggaaggccttggcctcccgcccggcgacgaccccatgctcctc
gcctacctcctggacccttccaacaccacccccgagggggtggcccgg
cgctacggcggggagtggacggaggaggcgggggagcgggccgccctt
tccgagaggctcttcgccaacctgtgggggaggcttgagggggaggag
aggctcctttggctttaccgggaggtggagaggcccctttccgctgtc
ctggcccacatggaggccacgggggtgcgcctggacgtggcctatctc
agggccttgtccctggaggtggccgaggagatcgcccgcctcgaggcc
gaggtcttccgcctggccggccaccccttcaacctcaactcccgggac
cagctggaaagggtcctctttgacgagctagggcttcccgccatcggc
aagacggagaagaccggcaagcgctccaccagcgccgccgtcctggag
gccctccgcgaggcccaccccatcgtggagaagatcctgcagtaccgg
gagctcaccaagctgaagagcacctacattgaccccttgccggacctc
atccaccccaggacgggccgcctccacacccgcttcaaccagacggcc
acggccacgggcaggctaagtagctccgatcccaacctccagaacatc
cccgtccgcaccccgcttgggcagaggatccgccgggccttcatcgcc
gaggaggggtggctattggtggccctggactatagccagatagagctc
agggtgctggcccacctctccggcgacgagaacctgatccgggtcttc
caggaggggcgggacatccacacggagaccgccagctggatgttcggc
gtcccccgggaggccgtggaccccctgatgcgccgggcggccaagacc
atcaacttcggggtcctctacggcatgtcggcccaccgcctctcccag
gagctagccatcccttacgaggaggcccaggccttcattgagcgctac
tttcagagcttccccaaggtgcgggcctggattgagaagaccctggag
gagggcaggaggcgggggtacgtggagaccctcttcggccgccgccgc
tacgtgccagacctagaggcccgggtgaagagcgtgcgggaggcggcc
gagcgcatggccttcaacatgcccgtccagggcaccgccgccgacctc
atgaagctggctatggtgaagctcttccccaggctggaggaaatgggg
gccaggatgctccttcaggtccacgacgagctggtcctcgaggcccca
aaagagagggcggaggccgtggcccggctggccaaggaggtcatggag
ggggtgtatcccctggccgtgcccctggaggtggaggtggggataggg
gaggactggctctccgccaaggagtga,

and a corresponding amino acid sequence:

(SEQ ID NO: 22)
MEEVLDREYEVEYGGRKYRLKPVKAWVLQPPGKPGVVIALFKLPDGKT
IRKVIMKLPPSVTHMGLLHEFGLLESPKALEEAPWPPPEGAFVGFVLS
RKEPMWADLLALAAARGGRVHRAPEPYKALRDLKEARGLLAKDLSVLA
LREGLGLPPGDDPMLLAYLLDPSNTTPEGVARRYGGEWTEEAGERAAL
SERLFANLWGRLEGEERLLWLYREVERPLSAVLAHMEATGVRLDVAYL
RALSLEVAEEIARLEAEVFRLAGHPFNLNSRDQLERVLFDELGLPAIG
KTEKTGKRSTSAAVLEALREAHPIVEKILQYRELTKLKSTYIDPLPDL
IHPRTGRLHTRFNQTATATGRLSSSDPNLQNIPVRTPLGQRIRRAFIA
EEGWLLVALDYSQIELRVLAHLSGDENLIRVFQEGRDIHTETASWMFG
VPREAVDPLMRRAAKTINFGVLYGMSAHRLSQELAIPYEEAQAFIERY
FQSFPKVRAWIEKTLEEGRRRGYVETLFGRRRYVPDLEARVKSVREAA
ERMAFNMPVQGTAADLMKLAMVKLFPRLEEMGARMLLQVHDELVLEAP
KERAEAVARLAKEVMEGVYPLAVPLEVEVGIGEDWLSAKE.

2. Construction of Cren7 Enhancer Domain-Taq_DNA Polymerase Fusion Proteins

The entire Ape Cren7 enhancer domain gene was amplified by PCR from genomic DNA using the following primer set:

Upper
(SEQ ID NO: 34)
5′-GATATCCATATGAGCCAGAAGCAACTACCA-3′
Lower
(SEQ ID NO: 40)
5′-ATCCCCGAATTCATGGTAACACCACCCCCGGTCTCGGGGTAGT
CG-3′.

The forward primer contained an Ndel site and reverse primers contained an EcoRI site. The reverse primer, in addition, coded for small linker peptide (sequence Gly-Gly-Val-Thr[SEQ ID NO: 41]) between the end of the Cren7 enhancer domain gene and the G of the EcoR I site.

The PCR reactions contained Pfu DNA polymerase reaction buffer, 200 μM each dNTP, 0.5 μM forward and reverse primers, 100 ng genomic DNA and 0.05 u/μl of a mixture of Taq and Pfu (20:1 ratio) DNA polymerases.

The cycling protocol was 94° C. for 60 s; 25 cycles of 94° C. for 10 s and 72° C. for 15 s.

The entire Sso Cren7 enhancer domain gene was similarly amplified by PCR from genomic DNA with the following primer set:

Upper
(SEQ ID NO: 36)
5′-GATATCCATATGAGTTCGGGTAAAAAACC-3′
Lower
(SEQ ID NO: 42)
5′-ATCCCCGAATTCATGGTAACACCACCTATTGGATAATCATCTG
GT-3′.

The entire Hbu Cren7 enhancer domain gene 1128 was similarly amplified by PCR from genomic DNA with the following primer set:

Upper
(SEQ ID NO: 43)
5′-GATATCCATATGGCGTGTGAGAAGCCTGTT-3′
Lower
SEQ ID NO: 44)
5′-ATCCCCGAATTCATGGTAACACCACCGCTGCAGATTGGGTAGT
CG-3′.

The amplified Cren7 enhancer domain gene was digested with NdeI and EcoRI cloned into an expression vector, pTTQ18TAQ, which encodes Taq DNA polymerase (see Engelke et al., 1990, cited above, for Taq DNA polymerase sequence). The ligated DNA was used to transform E. coli cells TOP10F′ and transformants plated on an Ampicillin plate. In plasmid mini-prep screening, approximately eight out of ten were found to contain the correct size insert.

E. coli cells carrying sequenced insert plasmid were induced by addition of IPTG for 4 hours. Cells were lysed by sonication. The clarified lysate was then heat treated at 70° C. for 30 min to inactivate the endogenous polymerases.

In more detail, a single colony was inoculated into 10 mls LB (+antibiotic at 50 μg/ml) and grown overnight at 37° C., with shaking at 275 rpm. 5 mls of this primary culture was transferred to a 2 litre capacity shakeflask containing 900 mls of TB and 100 mls TB salt solution. Antibiotic was added to 50 μg/ml. The culture was incubated at 37° C. (275 rpm) for ˜4 hrs (until a reading of OD₆₀₀1 was reached). IPTG (1 mM final) was added to the culture to induce protein expression for 4 hrs. Cells were harvested by centrifugation (2000×g for 15 mins) and frozen at −80° C.

(Autoclaved Luria Broth (LB): 10 g tryptone, 5 g yeast extract, 5 g NaCl, in 1 Litre dH₂O; Autoclaved Terrific Broth (TB): 12 g tryptone, 24 g yeast extract, 4 mls glycerol in 900 mls dH₂O;

Autoclaved TB Salt Solution: 0.17M KH₂PO₄, 0.72M K₂HPO₄)

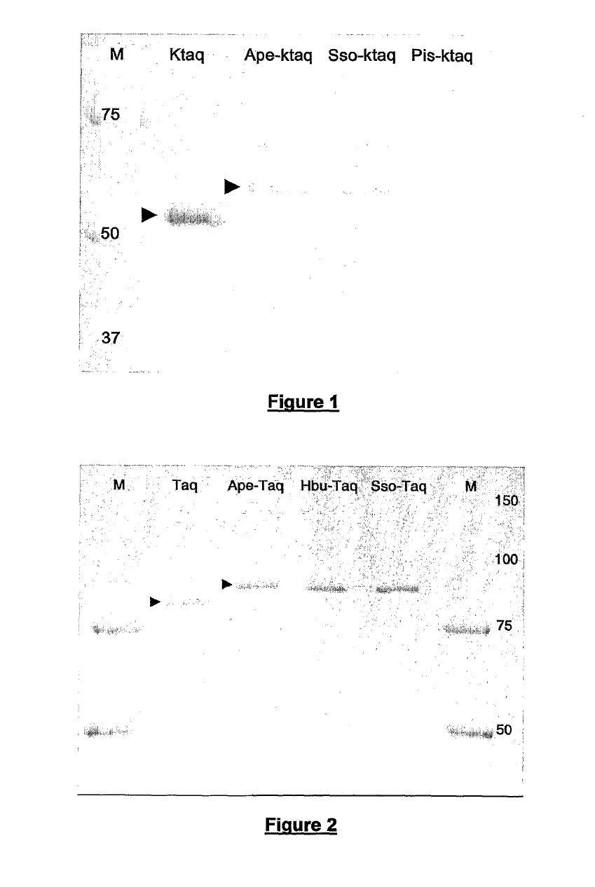

Cren7 enhancer domain-Taq DNA polymerase fusions (“Taq fusions”) were subjected to electrophoresis in a 8% SDS PAGE gel. A major band of about 94 kDa was detected as compared to 88 kDa for similarly induced non-fusion Taq DNA polymerase, as shown in FIG. 2. This correlates with the predicted molecular weights of around 101 kDa for the chimeric protein and around 93 kDa for the non-chimeric Taq DNA polymerase; DNA polymerases are known to sometimes run slightly faster than expected on SDS PAGE gels, so that their apparent molecular weight is smaller than predicted.

The Ape Cren7 enhancer domain-Taq fusion protein cloned as described above has the following DNA sequence (5′-3′):

(SEQ ID NO: 26)
atgagccagaagcaactaccacctgtgaaggtcagggacccgactaca
ggcaaggaggtcgagctaacgccaatcaaagtgtggaagctatcgccg
agggggaggaggggcgtcaagataggtctcttcaagagccccgagacg
ggcaagtacttcagggccaaggtgcccgacgactaccccgagaccggg
ggtggtgttaccatgaattcggggatgctgcccctctttgagcccaag
ggccgggtcctcctggtggacggccaccacctggcctaccgcaccttc
cacgccctgaagggcctcaccaccagccggggggagccggtgcaggcg
gtctacggcttcgccaagagcctcctcaaggccctcaaggaggacggg
gacgcggtgatcgtggtctttgacgccaaggccccctccttccgccac
gaggcctacggggggtacaaggcgggccgggcccccacgccggaggac
tttccccggcaactcgccctcatcaaggagctggtggacctcctgggg
ctggcgcgcctcgaggtcccgggctacgaggcggacgacgtcctggcc
agcctggccaagaaggcggaaaaggagggctacgaggtccgcatcctc
accgccgacaaagacctttaccagctcctttccgaccgcatccacgtc
ctccaccccgaggggtacctcatcaccccggcctggctttgggaaaag
tacggcctgaggcccgaccagtgggccgactaccgggccctgaccggg
gacgagtccgacaaccttcccggggtcaagggcatcggggagaagacg
gcgaggaagcttctggaggagtgggggagcctggaagccctcctcaag
aacctggaccggctgaagcccgccatccgggagaagatcctggcccac
atggacgatctgaagctctcctgggacctggccaaggtgcgcaccgac
ctgcccctggaggtggacttcgccaaaaggcgggagcccgaccgggag
aggcttagggctttctggagaggcttgagtttggcagcctcctccacg
agttcggccttctggaaagccccaaggccctggaggaggccccctggc
ccccgccggaaggggccttcgtgggctttgtgctttcccgcaaggagc
ccatgtgggccgatcttctggccctggccgccgccagggggggccggg
tccaccgggcccccgagccttataaagccctcagggacctgaaggagg
cgcgggggcttctcgccaaagacctgagcgttctggccctgagggaag
gccttggcctcccgcccggcgacgaccccatgctcctcgcctacctcc
tggacccttccaacaccacccccgagggggtggcccggcgctacgcgg
ggagtggacggaggaggcgggggagcgggccgccctttccgagaggct
cttcgccaacctgtgggggaggcttgagggggaggagaggctcctttg
gctttaccgggaggtggagaggcccctttccgctgtcctggcccacat
ggaggccacgggggtgcgcctggacgtggcctatctcagggccttgtc
cctggaggtggccgaggagatcgcccgcctcgaggcgaggtcttccgc
ctggccggccaccccttcaacctcaactcccgggaccagctggaaagg
gtcctctttgacgagctagggcttcccgccatcggcaagacggagaag
accggcaagcgctccaccagcgccgccgtcctggaggccctccgcgag
gcccaccccatcgtggagaagatcctgcagtaccgggagctcaccaag
ctgaagagcacctacattgaccccttgccggacctcatccaccccagg
acgggccgcctccacacccgcttcaaccagacggccacggccacgggc
aggctaagtagctccgatcccaacctccagaacatccccgtccgcacc
ccgcttgggcagaggatccgccgggccttcatcgccgaggaggggtgg
ctattggtggccctggactatagccagatagagctcagggtgctggcc
cacctctccggcgacgagaacctgatccgggtcttccaggaggggcgg
gacatccacacggagaccgccagctggatgttcggcgtcccccgggag
gccgtggaccccctgatgcgccgggcggccaagaccatcaacttcggg
gtcctctacggcatgtcggcccaccgcctctcccaggagctagccatc
ccttacgaggaggcccaggccttcattgagcgctactttcagagcttc
cccaaggtgcgggcctggattgagaagaccctggaggagggcaggagg
cgggggtacgtggagaccctcttcggccgccgccgctacgtgccagac
ctagaggcccgggtgaagagcgtgcgggaggcggccgagcgcatggcc
ttcaacatgcccgtccagggcaccgccgccgacctcatgaagctggct
atggtgaagctcttccccaggctggaggaaatgggggccaggatgctc
cttcaggtccacgacgagctggtcctcgaggccccaaaagagagggcg
gaggccgtggcccggctggccaaggaggtcatggagggggtgtatccc
ctggccgtgcccctggaggtggaggtggggataggggaggactggctc
tccgccaaggagtga,

and a corresponding amino acid sequence:

(SEQ ID NO: 17)
MSQKQLPPVKVRDPTTGKEVELTPIKVWKLSPRGRRGVKIGLFKSPET
GKYFRAKVPDDYPETGGGVTMDSGMLPLFEPKGRVLLVDGHHLAYRTF
HALKGLTTSRGEPVQAVYGFAKSLLKALKEDGDAVIVVFDAKAPSFRH
EAYGGYKAGRAPTPEDFPRQLALIKELVDLLGLARLEVPGYEADDVLA
SLAKKAEKEGYEVRILTADKDLYQLLSDRIHVLHPEGYLITPAWLWEK
YGLRPDQWADYRALTGDESDNLPGVKGIGEKTARKLLEEWGSLEALLK
NLDRLKPAIREKILAHMDDLKLSWDLAKVRTDLPLEVDFAKRREPDRE
RLRAFLERLEFGSLLHEFGLLESPKALEEAPWPPPEGAFVGFVLSRKE
PMWADLLALAAARGGRVHRAPEPYKALRDLKEARGLLAKDLSVLALRE
GLGLPPGDDPMLLAYLLDPSNTTPEGVARRYGGEWTEEAGERAALSER
LFANLWGRLEGEERLLWLYREVERPLSAVLAHMEATGVRLDVAYLRAL
SLEVAEEIARLEAEVFLAGHPFNLNSRDQLERVLFDELGLPAIGKTEK
TGKRSTSAAVLEALREAHPIVEKILQYRELTKLKSTYIDPLPDLIHPR
TGRLHTRFNQTATATGRLSSSDPNLQNIPVRTPLGQRIRRAFIAEEGW
LLVALDYSQIELRVLAHLSGDENLIRVFQEGRDIHTETASWMFGVPRE
AVDPLMRRAAKTINFGVLYGMSAHRLSQELAIPYEEAQAFIERYFQSF
PKVRAWIEKTLEEGRRRGYVETLFGRRRYVPDLEARVKSVREAAERMA
FNMPVQGTAADLMKLAMVKLFPRLEEMGARMLLQVHDELVLEAPKERA
EAVARLAKEVMEGVYPLAVPLEVEVGIGEDWLSAKE.

The Sso Cren7 enhancer domain-Taq fusion protein cloned as described above has the following nucleotide sequence (5′-3′):

(SEQ ID NO: 29)
atgagttcgggtaaaaaaccagtaaaagtaaaaacaccagctggtaaa
gaggctgaattggttccagaaaaagtatgggcattagcaccaaagggt
agaaaaggtgtaaagataggtttatttaaagatccagaaactgggaaa
tacttcagacataagctaccagatgattatccaataggtggtgttacc
atgaattcggggatgctgcccctctttgagcccaagggccgggtcctc
ctggtggacggccaccacctggcctaccgcaccttccacgccctgaag
ggcctcaccaccagccggggggagccggtgcaggcggtctacggcttc
gccaagagcctcctcaaggccctcaaggaggacggggacgcggtgatc
gtggtctttgacgccaaggccccctccttccgccacgaggcctacggg
gggtacaaggcgggccgggcccccacgccggaggactttccccggcaa
ctcgccctcatcaaggagctggtggacctcctggggctggcgcgcctc
gaggtcccgggctacgaggcggacgacgtcctggccagcctggccaag
aaggcggaaaaggagggctacgaggtccgcatcctcaccgccgacaaa
gacctttaccagctcctttccgaccgcatccacgtcctccaccccgag
gggtacctcatcaccccggcctggctttgggaaaagtacggcctgagg
cccgaccagtgggccgactaccgggccctgaccggggacgagtccgac
aaccttcccggggtcaagggcatcggggagaagacggcgaggaagctt
ctggaggagtgggggagcctggaagccctcctcaagaacctggaccgg
ctgaagcccgccatccgggagaagatcctggcccacatggacgatctg
aagctctcctgggacctggccaaggtgcgcaccgacctgcccctggag
gtggacttcgccaaaaggcgggagcccgaccgggagaggcttagggcc
tttctggagaggcttgagtttggcagcctcctccacgagttcggcctt
ctggaaagccccaaggccctggaggaggccccctggcccccgccggaa
ggggccttcgtgggctttgtgctttcccgcaaggagcccatgtgggcc
gatcttctggccctggccgccgccagggggggccgggtccaccgggcc
cccgagccttataaagccctcagggacctgaaggaggcgcgggggctt
ctcgccaaagacctgagcgttctggccctgagggaaggccttggcctc
ccgcccggcgacgaccccatgctcctcgcctacctcctggacccttcc
aacaccaccccgagggggtggcccggcgctacggcggggagtggacgg
aggaggcgggggagcgggccgccctttccgagaggctcttcgccaacc
tgtgggggaggcttgagggggaggagaggctcctttggctttaccggg
aggtggagaggcccctttccgctgtcctggcccacatggaggccacgg
gggtgcgcctggacgtggcctatctcagggccttgtccctggaggtgg
ccgaggagatcgcccgcctcgaggccgaggtcttccgcctggccggcc
accccttcaacctcaactcccgggaccagctggaaagggtcctctttg
acgagctagggcttcccgccatcggcaagacggagaagaccggcaagc
gctccaccagcgccgccgtcctggaggccctccgcgaggcccacccca
tcgtggagaagatcctgcagtaccgggagctcaccaagctgaagagca
cctacattgaccccttgccggacctcatccaccccaggacgggccgcc
tccacacccgcttcaaccagacggccacggccacgggcaggctaagta
gctccgatcccaacctccagaacatccccgtccgcaccccgcttgggc
agaggatccgccgggccttcatcgccgaggaggggtggctattggtgg
ccctggactatagccagatagagctcagggtgctggcccacctctccg
gcgacgagaacctgatccgggtcttccaggaggggcgggacatccaca
cggagaaccgccagctggatgttcggcgtcccccgggaggccgtggac
cccctgatgcgccgggcggccaagaccatcaacttcggggtcctctac
ggcatgtcggcccaccgcctctcccaggagctagccatcccttacgag
gaggcccaggccttcattgagcgctactttcagagcttccccaaggtg
cgggcctggattgagaagaccctggaggagggcaggaggcgggggtac
gtggagaccctcttcggccgccgccgctacgtgccagacctagaggcc
cgggtgaagagcgtgcgggaggcggccgagcgcatggccttcaacatg
cccgtccagggcaccgccgccgacctcatgaagctggctatggtgaag
ctcttccccaggctggaggaaatgggggccaggatgctccttcaggtc
cacgacgagctggtcctcgaggccccaaaagagagggcggaggccgtg
gcccggctggccaaggaggtcatggagggggtgtatcccctggccgtg
cccctggaggtggaggtggggataggggaggactggctctccgccaag
gagtga,

and a corresponding amino acid sequence:

(SEQ ID NO: 20)
MSSGKKPVKVKTPAGKEAELVPEKVWALAPKGRKGVKIGLFKDPETGK
YFRHKLPDDYPIGGVTMDSGMLPLFEPKGRVLLVDGHHLAYRTFHALK
GLTTSRGEPVQAVYGFAKSLLKALKEDGDAVIVVFDAKAPSFRHEAYG
GYKAGRAPTPEDFPRQLALIKELVDLLGLARLEVPGYEADDVLASLAK
KAEKEGYEVRILTADKDLYQLLSDRIHVLHPEGYLITPAWLWEKEKYG
LRPDQWADYRALTGDESDNLPGVKGIGEKTARKLLEEWGSLEALLKNL
DRLKPAIREKILAHMDDLKLSWDLAKVRTDLPLEVDFAKRREPDRERL
RAFLERLEFGSLLHEFGLLESPKALEEAPWPPPEGAFVGFVLSRKEPM
WADLLALAAARGGRVHRAPEPYKALRDLKEARGLLAKDLSVLALREGL
GLPPGDDPMLLAYLLDPSNTTPEGVARRYGGEWTEEAGERAALSERLF
ANLWGRLEGEERLLWLYREVERPLSAVLAHMEATGVRLDVAYLRALSL
EVAEEIARLEAEVFRLAGHPFNLNSRDQLERVLFDELGLPAIGKTEKT
GKRSTSAAVLEALREAHPIVEKILQYRELTKLKSTYIDPLPDLIHPRT
GRLHTRFNQTATATGRLSSSDPNLQNIPVRTPLGQRIRRAFIAEEGWL
LVALDYSQIELRVLAHLSGDENLIRVFQEGRDIHTETASWMFGVPREA
VDPLMRRAAKTINFGVLYGMSAHRLSQELAIPYEEAQAFIERYFQSFP
KVRAWIEKTLEEGRRRGYVETLFGRRRYVPDLEARVKSVREAAERMAF
NMPVQGTAADLMKLAMVKLFPRLEEMGARMLLQVHDELVLEAPKERAE
AVARLAKEVMEGVYPLAVPLEVEVGIGEDWLSAKE.

The Hbu Cren7 enhancer domain-Taq fusion protein cloned as described above has the following nucleotide sequence (5′-3′):

(SEQ ID NO: 32)
atggcgtgtgagaagcctgttaaggttcgtgaccctactactggtaag
gaggtagagctggtaccaatcaaggtgtggcagctagcacccaggggt
aggaagggcgtcaagataggcctattcaagagccccgaaacaggcaag
tacttcagagccaaggtaccagacgactacccaatctgcagcggtggt
gttaccatgaattcggggatgctgcccctctttgagcccaagggccgg
gtcctcctggtggacggccaccacctggcctaccgcaccttccacgcc
ctgaagggcctcaccaccagccggggggagccggtgcaggcggtctac
ggcttcgccaagagcctcctcaaggccctcaaggaggacggggacgcg
gtgatcgtggtctttgacgccaaggccccctccttccgccacgaggcc
tacggggggtacaaggcgggccgggcccccacgccggaggactttccc
cggcaactcgccctcatcaaggagctggtggacctcctggggctggcg
cgcctcgaggtcccgggctacgaggcggacgacgtcctggccagcctg
gccaagaaggcggaaaaggagggctacgaggtccgcatcctcaccgcc
gacaaagacctttaccagctcctttccgaccgcatccacgtcctccac
cccgaggggtacctcatcaccccggcctggctttgggaaaagtacggc
ctgaggcccgaccagtgggccgactaccgggccctgaccggggacgag
tccgacaaccttcccggggtcaagggcatcggggagaagacggcgagg
aagcttctggaggagtgggggagcctggaagccctcctcaagaacctg
gaccggctgaagcccgccatccgggagaagatcctggcccacatggac
gatctgaagctctcctgggacctggccaaggtgcgcaccgacctgccc
ctggaggtggacttcgccaaaaggcgggagcccgaccgggagaggctt
agggcctttctggagaggcttgagtttggcagcctcctccacgagttc
ggccttctggaaagccccaaggccctggaggaggccccctggcccccg
ccggaaggggccttcgtgggctttgtgctttcccgcaaggagcccatg
tgggccgatcttctggccctggccgccgccagggggggccgggtccac
cgggcccccgagccttataaagccctcagggacctgaaggaggcgcgg
gggcttctcgccaaagacctgagcgttctggccctgagggaaggcctt
ggcctcccgcccggcgacgaccccatgctcctcgcctacctcctggac
ccttccaacaccaccccgagggggtggcccggcgctacggcggggagt
ggacggaggaggcgggggagcgggccgccctttccgagaggctcttcg
ccaacctgtgggggaggcttgagggggaggagaggctcctttggcttt
accgggaggtggagaggcccctttccgctgtcctggcccacatggagg
ccacgggggtgcgcctggacgtggcctatctcagggccttgtccctgg
aggtggccgaggagatcgcccgcctcgaggccgaggtcttccgcctgg
ccggccaccccttcaacctcaactcccgggaccagctggaaagggtcc
tctttgacgagctagggcttcccgccatcggcaagacggagaagaccg
gcaagcgctccaccagcgccgccgtcctggaggccctccgcgaggccc
accccatcgtggagaagatcctgcagtaccgggagctcaccaagctga
agagcacctacattgaccccttgccggacctcatccaccccaggacgg
gccgcctccacacccgcttcaaccagacggccacggccacgggcaggc
taagtagctccgatcccaacctccagaacatccccgtccgcaccccgc
ttgggcagaggatccgccgggccttcatcgccgaggaggggtggctat
tggtggccctggactatagccagatagagctcagggtgctggcccacc
tctccggcgacgagaacctgatccgggtcttccaggaggggcgggaca
tccacacggagaccgccagctggatgttcggcgtcccccgggaggccg
tggaccccctgatgcgccgggcggccaagaccatcaacttcggggtcc
tctacggcatgtcggcccaccgcctctcccaggagctagccatccctt
acgaggaggcccaggccttcattgagcgctactttcagagcttcccca
aggtgcgggcctggattgagaagaccctggaggagggcaggaggcggg
ggtacgtggagaccctcttcggccgccgccgctacgtgccagacctag
aggcccgggtgaagagcgtgcgggaggcggccgagcgcatggccttca
acatgcccgtccagggcaccgccgccgacctcatgaagctggctatgg
tgaagctcttccccaggctggaggaaatgggggccaggatgctccttc
aggtccacgacgagctggtcctcgaggccccaaaagagagggcggagg
ccgtggcccggctggccaaggaggtcatggagggggtgtatcccctgg
ccgtgcccctggaggtggaggtggggataggggaggactggctctccg
ccaaggagtga,

and a corresponding amino acid sequence:

(SEQ ID NO: 23)
MACEKPVKVRDPTTGKEVELVPIKVWQLAPRGRKGVKIGLFKSPETGK
YFRAKVPDDYPICSGGVTMDSGMLPLFEPKGRVLLVDGHHLAYRTFHA
LKGLTTSRGEPVQAVYGFAKSLLKALKEDGDAVIVVFDAKAPSFRHEA
YGGYKAGRAPTPEDFPRQLALIKELVDLLGLARLEVPGYEADDVLASL
AKKAEKEGYEVRILTADKDLYQLLSDRIHVLHPEGYLITPAWLWEKYG
LRPDQWADYRALTGDESDNLPGVKGIGEKTARKLLEEWGSLEALLKNL
DRLKPAIREKILAHMDDLKLSWDLAKVRTDLPLEVDFAKRREPDRERL
RAFLERLEFGSLLHEFGLLESPKALEEAPWPPPEGAFVGFVLSRKEPM
WADLLALAAARGGRVHRAPEPYKALRDLKEARGLLAKDLSVLALREGL
GLPPGDDPMLLAYLLDPSNTTPEGVARRYGGEWTEEAGERAALSERLF
ANLWGRLEGEERLLWLYREVERPLSAVLAHMEATGVRLDVAYLRALSL
EVAEEIARLEAEVFRLAGHPFNLNSRDQLERVLFDELGLPAIGKTEKT
GKRSTSAAVLEALREAHPIVEKILQYRELTKLKSTYIDPLPDLIHPRT
GRLHTRFNQTATATGRLSSSDPNLQNIPVRTPLGQRIRRAFIAEEGWL
LVALDYSQIELRVLAHLSGDENLIRVFQEGRDIHTETASWMFGVPREA
VDPLMRRAAKTINFGVLYGMSAHRLSQELAIPYEEAQAFIERYFQSFP
KVRAWIEKTLEEGRRRGYVETLFGRRRYVPDLEARVKSVREAAERMAF
NMPVQGTAADLMKLAMVKLFPRLEEMGARMLLQVHDELVLEAPKERAE
AVARLAKEVMEGVYPLAVPLEVEVGIGEDWLSAKE.

3. Construction of Cren7 Enhancer Domain-Pfu DNA Polymerase Proteins

The entire Ape Cren7 enhancer domain gene was amplified by PCR from genomic DNA using the following primer set:

Upper
(SEQ ID NO: 45)
5′-GAATTCGGTACCCATAGCCAGAAGCAACTA-3′
Lower
(SEQ ID NO: 46)
5′-GAATTCGTCGACTTACCCGGTCTCGGGGTA-3′.

The forward primer contained a KpnI site and reverse primers contained a stop codon followed by a SalI site.

The PCR reactions contained Pfu DNA polymerase reaction buffer, 200 μM each dNTP, 0.5μM forward and reverse primers, 100 ng genomic DNA and 0.05 u/μl of a mixture of Taq and Pfu (20:1 ratio) DNA polymerases.

The cycling protocol was 94° C. for 60 s; 25 cycles of 94° C. for 10 s and 72° C. for 15 s.

The entire Sso Cren7 enhancer domain gene was similarly amplified by PCR from genomic DNA with the following primer set:

Upper
(SEQ ID NO: 47)
5′-GAATTCGGTACCCATATGAGTTCGGGTAAA-3′
Lower
(SEQ ID NO: 48)
5′-GAATTCGTCGACTTATATTGGATAATCATC-3′.

The entire Hbu Cren7 enhancer domain gene was similarly amplified by PCR from genomic DNA with the following primer set:

Upper
(SEQ ID NO: 49)
5′-GAATTCGGTACCCATATGGCGTGTGAGAAG-3′
Lower
(SEQ ID NO: 50)
5′-GAATTCGTCGACTTAGCTGCAGATTGGGTA-3′.

Plasmid pET21 a (Novagen) was used to produce plasmid pET21aPFU carrying the Pfu DNA polymerase gene (see Lu & Erickson, 1997, Protein Expr. Purif. 11: 179-184) under the control of the T7 promoter. This pET21aPFU plasmid was modified to introduce a unique restriction site at the 3′ end of the Pfu gene. The resulting plasmid (pET21aPfuKpn) expresses a Pfu polymerase (PfuKpn) with three additional amino acids (Gly-Thr-His) at its C-terminus. No functional difference was observed between PfuKpn and commercial Pfu polymerase (Stratagene).

The amplified Cren7 enhancer domain genes were digested with KpnI and SalI cloned into the expression vector, pET21aPfuKpn. The ligated DNA was used to transform E.coli cells TOP10F′ and transformants plated on an Ampicillin plate. In plasmid mini-prep screening, approximately eight out of ten was found to contain the correct size insert for Ape and Sso constructs.

Only one Hbu correct construct was found due to inadvertently missing the presence of two internal KpnI sites within the gene.

Plasmids from single sequenced clones were isolated and transformed into E. coli BL21(DE3)pLYSS (Novagen; see also Studier et al., 1990, Methods Enzymol. 185: 60-89 and U.S. Pat. No. 4,952,496). E. coli cells carrying insert plasmid were induced by addition of IPTG for 4 hours. Cells were lysed by sonication. The clarified lysate was then heat treated at 70° C. for 30 min to inactivate the endogenous polymerases.

In more detail, a single colony was inoculated into 10 mls LB (+antibiotic at 50 μg/ml) and grown overnight at 37° C., with shaking at 275 rpm. 5mls of this primary culture was transferred to a 2 litre capacity shake flask containing 900 mls of TB and 100 mls TB salt solution. Antibiotic was added to 50 ug/ml. The culture was incubated at 37° C. (275rpm) for ˜4 hrs (until a reading of OD₆₀₀1.0 was reached). IPTG (1 mM final) was added to the culture to induce protein expression for 4 hrs. Cells were harvested by centrifugation (2000×g for 15 mins) and frozen at ˜80° C.

(Autoclaved Luria Broth (LB): 10 g tryptone, 5 g yeast extract, 5 g NaCl, in 1 Litre dH₂O;

Autoclaved Terrific Broth (TB): 12 g tryptone, 24 g yeast extract, 4 mls glycerol in 900 mls dH₂O;

Autoclaved TB Salt Solution: 0.17M KH₂PO₄, 0.72M K₂HPO₄)

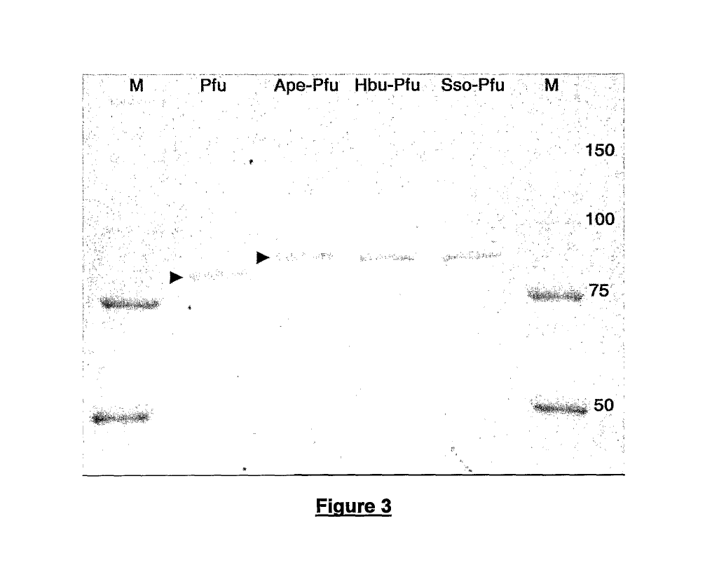

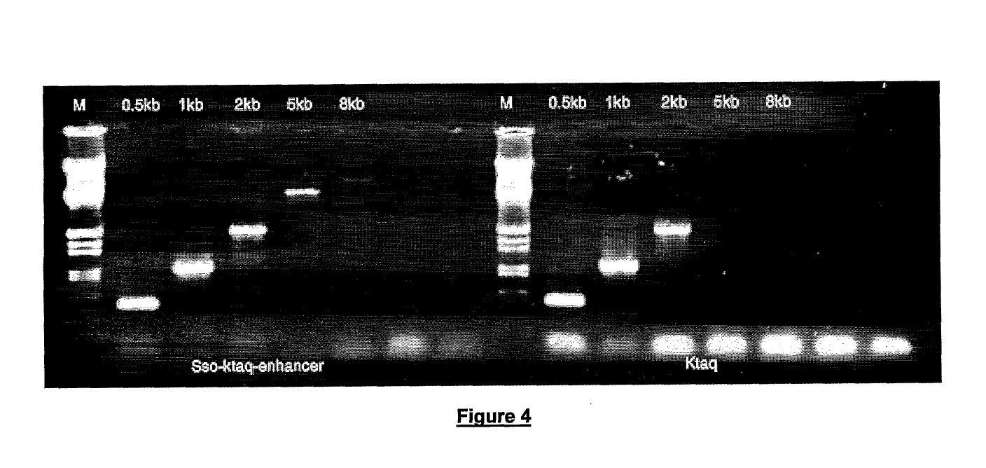

Cren7 enhancer domain-Pfu DNA polymerase fusions (“Pfu fusions”) were subjected to electrophoresis in an 8% SDS PAGE gel. A major band of about 96 kDa was detected as compared to 88 kDa for similarly induced non-fusion Pfu DNA polymerase, as shown in FIG. 3. This correlates with the predicted molecular weights of around 97 kDa for the chimeric protein and around 89 kDa for the non-chimeric Pfu DNA polymerase; DNA polymerases are known to sometimes run slightly faster than expected on SDS PAGE gels, so that their apparent molecular weight is smaller than predicted.

The Ape Cren7 enhancer domain-Pfu fusion protein cloned as described above has the following nucleotide sequence (5′-3′):

(SEQ ID NO: 27)
atgattttagatgtggattacataactgaagaaggaaaacctgttatt
aggctattcaaaaaagagaacggaaaatttaagatagagcatgataga
acttttagaccatacatttacgctcttacagggatgattcaaagattg
aagaagttaagaaaataacgggggaaaggcatggaaagattgtgagaa
ttgttgatgtagagaaggttgagaaaaagtttctcggcaagcctatta
ccgtgtggaaactttatttggaacatccccaagatgttcccactatta
gagaaaaagttagagaacatccagcagttgtggacatcttcgaatacg
atattccatttgcaaagagatacctcatcgacaaaggcctaataccaa
tggagggggaagaagagctaaagattatgccttcgatatagaaaccct
ctatcacgaaggagaagagtttggaaaaggcccaattataatgattag
ttatgcagatgaaaatgaagcaaaggtgattacttggaaaaacataga
tcttccatacgttgaggttgtatcaagcgagagagagatgataaagag
atttctcaggattatcagggagaaggatcctgacattatagttactta
taatggagactcattcgacttcccatatttagcgaaaagggcagaaaa
acttgggattaaattaaccattggaagagatggaagcgagcccaagat
gcagagaataggcgatatgacggctgtagaagtcaagggaagaataca
tttcgacttgtatcatgtaataacaaggacaataaatacccaacatac
acactagaggctgtatatgaagcaatttttggaaagccaaaggagaag
gtatacgccgacgagatagcaaaagcctgggaaagtggagagaacctt
gagagagttgccaaatactcgatggaagatgcaaaggcaacttatgaa
ctcgggaaagaattccttccaatggaaattcagctttcaagattagtt
tggacaaccttatgggatgtttcaaggtcaagcacagggaaccttgta
gagtggttcttacttaggaaagcctacgaaagaaacgaagtagctcca
aacaagccaagtgaagaggagtatcaaagaaggctcagggagagctac
acaggtggattcgttaaagagccagaaaaggggttgtgggaaaacata
gtatacctagattttagagccctatatccctcgattataattacccac
aatgtttctcccgatactctaaatcttgagggatgcaagaactatgat
atcgctcctcaagtaggccacaagttctgcaaggacatccctggtttt
ataccaagtctcttgggacatttgttagaggaaagacaaaagattaag
acaaaaatgaaggaaactcaagatcctatagaaaaaatactccttgac
tatagacaaaaagcgataaaactcttagcaaattctttctacggatat
tatggctatgcaaaagcaagatggtactgtaaggagtgtgctgagagc
gttactgcctggggaagaaagtacatcgagttagtatggaaggagctc
gaagaaaagtttggatttaaagtcctctacattgacactgatggtctc
tatgcaactatcccaggaggagaaagtgaggaaataaagaaaaaggct
ctagaatttgtaaaatacataaattcaaagctccctggactgctagag
cttgaatatgaagggttttataagaggggattcttcgttacgaagaag
aggtatgcagtaatagatgaagaaggaaaagtcattactcgtggttta
gagatagttaggagagattggagtgaaattgcaaaagaaactcaagct
agagttttggagacaatactaaaacacggagatgttgaagaagctgtg
agaatagtaaaagaagtaatacaaaagcttgccaattatgaaattcca
ccagagaagctcgcaatatatgagcagataacaagaccattacatgag
tataaggcgataggtcctcacgtagctgttgcaaagaaactagctgct
aaaggagttaaaataaagccaggaatggtaattggatacatagtactt
gatcccaaaaagcacaagtatgacgcagaatattacattgagaactag
aggcgaggtccaattagcaatagggcaattctagctgaggaataccag
gttatccagcggtacttaggatattggagggatttggatacagaaagg
aagacctcagataccaaaagacaagacaagtcggcctaacttcctggc
ttaacattaaaaaatccggtacccatagccagaagcaactaccacctg
tgaaggtcagggacccgactacaggcaaggaggtcgagctaacgccaa
tcaaagtgtggaagctatcgccgagggggaggaggggcgtcaagatag
gtctcttcaagagccccgagacgggcaagtacttcagggccaaggtgc
ccgacgactaccccgagaccgggtaa,

and a corresponding amino acid sequence:

(SEQ ID NO: 18)
MILDVDYITEEGKPVIRLFKKENGKFKIEHDRTFRPYIYALLRDDSKI
EEVKKITGERHGKIVRIVDVEKVEKKFLGKPITVWKLYLEHPQDVPTI
REKVREHPAVVDIFEYDIPFAKRYLIDKGLIPMEGEEELKILAFDIET
LYHEGEEFGKGPIIMISYADENEAKVITWKNIDLPYVEVVSSEREMIK
RFLRIIREKDPDIIVTYNGDSFDFPYLAKRAEKLGIKLTIGRDGSEPK
MQRIGDMTAVEVKGRIHFDLYHVITRTINLPTYTLEAVYEAIFGKPKE
KVYADEIAKAWESGENLERVAKYSMEDAKATYELGKEFLPMEIQLSRL
VGQPLWDVSRSSTGNLVEWFLLRKAYERNEVAPNKPSEEEYQRRLRES
YTGGFVKEPEKGLWENIVYLDFRALYPSIIITHNVSPDTLNLEGCKNY
DIAPQVGHKFCKDIPGFIPSLLGHLLEERQKIKTKMKETQDPIEKILL
DYRQKAIKLLANSFYGYYGYAKARWYCKECAESVTAWGRKYIELVWKE
LEEKFGFKVLYIDTDGLYATIPGGESEEIKKKALEFVKYINSKLPGLL
ELEYEGFYKRGFFVTKKRYAVIDEEGKVITRGLEIVRRDWSEIAKETQ
ARVLETILKHGDVEEAVRIVKEVIQKLANYEIPPEKLAIYEQITRPLH
EYKAIGPHVAVAKKLAAKGVKIKPGMVIGYIVLRGDGPISNRAILAEE
YDPKKHKYDAEYYIENQVLPAVLRILEGFGYRKEDLRYQKTRQVGLTS
WLNIKKSGTHSQKQLPPVKVRDPTTGKEVELTPIKVWKLSPRGRRGVK
IGLFKSPETGKYFRAKVPDDYPETG.

The Sso Cren7 enhancer domain-Pfu fusion protein cloned as described above has the following DNA sequence (5′-3′):

(SEQ ID NO: 30)
atgattttagatgtggattacataactgaagaaggaaaacctgttatt
aggctattcaaaaaagagaacggaaaatttaagatagagcatgataga
acttttagaccatacatttacgctcttctcagggatgattcaaagatt
gaagaagttaagaaaataacgggggaaaggcatggaaagattgtgaga
attgttgatgtagagaaggttgagaaaaagtttctcggcaagcctatt
accgtgtggaaactttatttggaacatccccaagatgttcccactatt
agagaaaaagttagagaacatccagcagttgtggacatcttcgaatac
gatattccatttgcaaagagatacctcatcgacaaaggcctaatacca
atggagggggaagaagagctaaagattcttgccttcgatatagaaacc
ctctatcacgaaggagaagagtttggaaaaggcccaattataatgatt
agttatgcagatgaaaatgaagcaaaggtgattacttggaaaaacata
gatcttccatacgttgaggttgtatcaagcgagagagagatgataaag
agatttctcaggattatcagggagaaggatcctgacattatagttact
tataatggagactcattcgacttcccatatttagcgaaaagggcagaa
aaacttgggattaaattaaccattggaagagatggaagcgagcccaag
atgcagagaataggcgatatgacggctgtagaagtcaagggaagaata
catttcgacttgtatcatgtaataacaaggacaataaatctcccaaca
tacacactagaggctgtatatgaagcaatttttggaaagccaaaggag
aaggtatacgccgacgagatagcaaaagcctgggaaagtggagagaac
cttgagagagttgccaaatactcgatggaagatgcaaaggcaacttat
gaactcgggaaagaattccttccaatggaaattcagctttcaagatta
gttggacaacctttatgggatgtttcaaggtcaagcacagggaacctt
gtagagtggttatacttaggaaagcctacgaaagaaacgaagtagctc
caaacaagccaagtgaagaggagtatcaaagaaggctcagggagagct
acacaggtggattcgttaaagagccagaaaaggggttgtgggaaaaca
tagtatacctagattttagagccctatatccctcgattataattaccc
acaatgtttctcccgatactctaaatcttgagggatgcaagaactatg
atatcgctcctcaagtaggccacaagttctgcaaggacatccctggtt
ttataccaagtctcttgggacatttgttagaggaaagacaaaagatta
agacaaaaatgaaggaaactcaagatcctatagaaaaaatactccttg
actatagacaaaaagcgataaaactcttagcaaattctttctacggat
attatggctatgcaaaagcaagatggtactgtaaggagtgtgctgaga
gcgttactgcctggggaagaaagtacatcgagttagtatggaaggagc
tcgaagaaaagtttggatttaaagtcctctacattgacactgatggtc
tctatgcaactatcccaggaggagaaagtgaggaaataaagaaaaagg
ctctagaatttgtaaaatacataaattcaaagctccctggactgctag
agcttgaatatgaagggttttataagaggggattcttcgttacgaaga
agaggtatgcagtaatagatgaagaaggaaaagtcattactcgtggtt
tagagatagttaggagagattggagtgaaattgcaaaagaaactcaag
ctagagttttggagacaatactaaaacacggagatgttgaagaagctg
tgagaatagtaaaagaagtaatacaaaagcttgccaattatgaaattc
caccagagaagctcgcaatatatgagcagataacaagaccattacatg
agtataaggcgataggtcctcacgtagctgttgcaaagaaactagctg
ctaaaggagttaaaataaagccaggaatggtaattggatacatagtac
ttagaggcgatggtccaattagcaatagggcaattctagctgaggaat
acgatcccaaaaagcacaagtatgacgcagaatattacattgagaacc
aggttcttccagcggtacttaggatattggagggatttggatacagaa
aggaagacctcagataccaaaagacaagacaagtcggcctaacttcct
ggcttaacattaaaaaatccggtacccatatgagttcgggtaaaaaac
cagtaaaagtaaaaacaccagctggtaaagaggctgaattggttccag
aaaaagtatgggcattagcaccaaagggtagaaaaggtgtaaagatag
gtttatttaaagatccagaaactgggaaatacttcagacataagctac
cagatgattatccaatataa,

and a corresponding amino acid sequence:

(SEQ ID NO: 21)
MILDVDYITEEGKPVIRLFKKENGKFKIEHDRTFRPYIYALLRDDSKI
EEVKKITGERHGKIVRIVDVEKVEKKFLGKPITVWKLYLEHPQDVPTI
REKVREHPAVVDIFEYDIPFAKRYLIDKGLIPMEGEEELKILAFDIET
LYHEGEEFGKGPIIMISYADENEAKVITWKNIDLPYVEVVSSEREMIK
RFLRIIREKDPDIIVTYNGDSFDFPYLAKRAEKLGIKLTIGRDGSEPK
MQRIGDMTAVEVKGRIHFDLYHVITRTINLPTYTLEAVYEAIFGKPKE
KVYADEIAKAWESGENLERVAKYSMEDAKATYELGKEFLPMEIQLSRL
VGQPLWDVSRSSTGNLVEWFLLRKAYERNEVAPNKPSEEEYQRRLRES
YTGGFVICEPEKGLWENIVYLDFRALYPSIIITHNVSPDTLNLEGCKN
YDIAPQVGHKFCKDIPGFIPSLLGHLLEERQKIKTKMKETQDPIEKIL
LDYRQKAIKLLANSFYGYYGYAKARWYCKECAESVTAWGRKYIELVWK
ELEEKFGFKVLYIDTDGLYATIPGGESEEIKKKALEFVKYINSKLPGL
LELEYEGFYKRGFFVTKKRYAVIDEEGKVITRGLEIVRRDWSEIAKET
QARVLETILKHGDVEEAVRIVKEVIQKLANYEIPPEKLAIYEQITRPL
HEYKAIGPHVAVAKKLAAKGVKIKPGMVIGYIVLRGDGPISNRAILAE
EYDPKKHKYDAEYYIENQVLPAVLRILEGFGYRKEDLRYQKTRQVGLT
SWLNIKKSGTHMSSGKKPVKVKTPAGKEAELVPEKVWALAPKGRKGVK
IGLFKDPETGKYFRHKLPDDYPI.

The Hbu Cren7 enhancer domain-Pfu fusion protein cloned as described above has the following DNA sequence (5′-3′):

(SEQ ID NO: 33)
atgattttagatgtggattacataactgaagaaggaaaacctgttatt
aggctattcaaaaaagagaacggaaaatttaagatagagcatgataga
acttttagaccatacatttacgctcttctcagggatgattcaaagatt
gaagaagttaagaaaataacgggggaaaggcatggaaagattgtgaga
attgttgatgtagagaaggttgagaaaaagtttctcggcaagcctatt
accgtgtggaaactttatttggaacatccccaagatgttcccactatt
agagaaaaagttagagaacatccagcagttgtggacatcttcgaatac
gatattccatttgcaaagagatacctcatcgacaaaggcctaatacca
atggagggggaagaagagctaaagattcttgccttcgatatagaaacc
ctctatcacgaaggagaagagtttggaaaaggcccaattataatgatt
agttatgcagatgaaaatgaagcaaaggtgattacttggaaaaacata
gatcttccatacgttgaggttgtatcaagcgagagagagatgataaag
agatttctcaggattatcagggagaaggatcctgacattatagttact
tataatggagactcattcgacttcccatatttagcgaaaagggcagaa
aaacttgggattaaattaaccattggaagagatggaagcgagcccaag
atgcagagaataggcgatatgacggctgtagaagtcaagggaagaata
catttcgacttgtatcatgtaataacaaggacaataaatctcccaaca
tacacactagaggctgtatatgaagcaatttttggaaagccaaaggag
aaggtatacgccgacgagatagcaaaagcctgggaaagtggagagaac
cttgagagagttgccaaatactcgatggaagatgcaaaggcaacttat
gaactcgggaaagaattccttccaatggaaattcagctttcaagatta
gttggacaacctttatgggatgtttcaaggtcaagcacagggaacctt
gtagagtggttcttacttaggaaagcctacgaaagaaacgaagtagct
ccaaacaagccaagtgaagaggagtatcaaagaaggctcagggagagc
tacacaggtggattcgttaaagagccagaaaaggggttgtgggaaaac
atagtatacctagattttagagccctatatccctcgattataattacc
cacaatgtttacccgatactctaaatcttgagggatgcaagaactatg
atatcgctcctcaagtaggccacaagttctgcaaggacatccctggtt
ttataccaagtctcttgggacatttgttagaggaaagacaaaagatta
agacaaaaatgaaggaaactcaagatcctatagaaaaaatactccttg
actatagacaaaaagcgataaaactcttagcaaattctttctacggat
ttaatggctatgcaaaagcaagatggtactgtaaggagtgtgctgaga
gcgttactgcctggggaagaaagtacatcgagttagtatggaaggagc
tcgaagaaaagtttggatttaaagtcctctacattgacactgatggtc
tctatgcaactatcccaggaggagaaagtgaggaaataaagaaaaagg
ctctagaatttgtaaaatacataaattcaaagctccctggactgctag
agcttgaatatgaagggttttataagaggggattcttcgttacgaaga
agaggtatgcagtaatagatgaagaaggaaaagtcattactcgtggtt
tagagatagttaggagagattggagtgaaattgcaaaagaaactcaag
ctagagttttggagacaatactaaaacacggagatgttgaagaagctg
tgagaatagtaaaagaagtaatacaaaagcttgccaattatgaaattc
caccagagaagctcgcaatatatgagcagataacaagaccattacatg
agtataaggcgataggtcctcacgtagctgttgcaaagaaactagctg
ctaaaggagttaaaataaagccaggaatggtaattggatacatagtac
ttagaggcgatggtccaattagcaatagggcaattctagctgaggaat
acgatcccaaaaagcacaagtatgacgcagaatattacattgagaacc
aggttcttccagcggtacttaggatattggagggatttggatacagaa
aggaagacctcagataccaaaagacaagacaagtcggcctaacttcct
ggcttaacattaaaaaatccggtacccatatggcgtgtgagaagcctg
ttaaggttcgtgaccctactactggtaaggaggtagagctggtaccaa
tcaaggtgtggcagctagcacccaggggtaggaagggcgtcaagatag
gcctattcaagagccccgaaacaggcaagtacttcagagccaaggtac
cagacgactacccaatctgcagctaa,

and a corresponding amino acid sequence:

(SEQ ID NO: 24)
MILDVDYITEEGKPVIRLFKKENGKFKIEHDRTFRPYIYALLRDDSKI
EEVKKITGERHGKIVRIVDVEKVEKKFIGKPITVWKLYLEHPQDVPTI
REKVREHPAVVDIFEYDIPFAKRYLIDKGLIPMEGEEELKILAFDIET
LYHEGEEFGKGPIIMISYADENEAKVITWKNIDLPYVEVVSSEREMIK
RFLRIIREKDPDIIVTYNGDSFDFPYLAKRAEKLGIKLTIGRDGSEPK
MQRIGDMTAVEVKGRIHFDLYHVITRTINLPTYTLEAVYEAIFGKPKE
KVYADEIAKAWESGENLERVAKYSMEDAKATYELGKEFLPMEIQLSRL
VGQPLWDVSRSSTGNLVEWFLLRKAYERNEVAPNKPSEEEYQRRLRES
YTGGFVKEPEKGLWENIVYLDFRALYPSIIITHNVSPDTLNLEGCKNY
DIAPQVGHKFCKDIPGFIPSLLGHLLEERQKIKTKMKETQDPIEKILL
DYRQKAIKLLANSFYGYYGYAKARWYCKECAESVTAWGRKYIELVWKE
LEEKFGFKVLYIDTDGLYATIPGGESEEIKKKALEFVKYINSKLPGLL
ELEYEGFYKRGFFVTKKRYAVIDEEGKVITRGLEIVRRDWSEIAKETQ
ARVLETILKHGDVEEAVRIVKEVIQKLANYEIPPEKLAIYEQITRPLH
EYKAIGPHVAVAKKLAAKGVKIKPGMVIGYIVLRGDGPISNRAILAEE
YDPKKHKYDAEYYIENQVLPAVLRILEGFGYRKEDLRYQKTRQVGLTS
WLNIKKSGTHMACEKPVKVRDPTTGKEVELVPIKVWQLAPRGRKGVK
IGLFKSPETGKYFRAKVPDDYPICS.

4. Purification of DNA Polymerases-Cren7 Enhancer Domain Fusions.

5 ml of an overnight culture was inoculated into 1 litre of LB +antibiotic (50 μg/ml). After incubation at 37° C. until an OD600 of 1.0, IPTG was added to 1 mM final concentration to induce the Cren7 enhancer domain-DNA polymerase fusion production. After IPTG induction for 4 hours at 37° C., cells were harvested by centrifugation at 5000 rpm for 15 mM. Cell pellets were resuspended in lysis buffer. Cells were lysed by sonication. The lysate was then heat treated at 75° C. for 30 mins, cooled to 4° C. and polyethyleneimine added to 1% final concentration. Cell debris, heat denatured proteins and precipitated nucleic acids were removed by centrifugation at 20,000 g for 30 min. The solution was then dialysed against 20 mM Tris-HCl pH7.5 and 50 mM NaCl. The proteins were loaded onto a Heparin Sepharose® column, unbound proteins washed off and the polymerase eluted with a NaCl gradient. Elution of the Cren7 enhancer domain-DNA polymerase fusions occurred at approximately 0.3M NaCl, 0.02M Tris-HCl.

The purified Cren7 enhancer domain-DNA polymerase fusions were subjected to electrophoresis in a 8% SDS PAGE gel. A single band of about 70 kDa for each of the corresponding fusions was detected as compared to 62 kDa for KlenTaq alone, 89 kDa for each of the corresponding fusions as compared to 83 kDa for Pfu alone, and 100 kDa for each of the corresponding fusions as compared to 94 kDa for Taq alone.

5. Further Method of Purification of DNA Polymerases-Cren7 Enhancer Fusions

All steps were performed at 4° C.

1 litre's worth of —80° C. frozen cell pastes were resuspended in 50 ml of Lysis Buffer (50 mM Trizma, 2 mM EDTA, 50 mM NaCl, pH 8.0) and made 0.15 mg/ml lysozyme). After 30 mM at 4° C. brought to 100 ml total volume with Lysis Buffer in 100 ml blue capped bottle. Lysate was sonicated for 2 mins to reduce viscosity.

Sodium chloride was added to a final concentration of 0.25M (1.5 g) and placed in an 80° C. pre-heated water bath and brought to temperature (approx 15 mins).

The mixture was held at 80° C. for an additional 45 min to precipitate host proteins.

The heat treated lysate was cooled on ice and 10% polyethyleneimine was added to a final concentration of 0.3%.

After overnight incubation, cell debris, heat denatured proteins and polymin P precipitated nucleic acids were pelleted at 10,000 rpm for 30 min at 4° C.

5 μl loaded onto 8% SDS protein gel.

AmmSO₄ was added to 70% to precipitate the enzyme and left overnight at 4° C. The mixture was centrifuged at 10,000 rpm for 30 mins, the pellet re-suspended in 10 ml buffer and dialysed against same buffer overnight. Any precipitate was removed by centrifugation at 10,000×g for 10 minutes and the supernatant was loaded onto Heparin column in 20 mM Trizma (pH 8.0), 1 mM EDTA, 0.05% Tween-20, 0.1M NaCl.

After washing with Column Buffer plus 100 mM NaCl until the A₂₈₀ returned to background, the enzyme was eluted from the Heparin-Sepharose column using a 10 CV 0-60% linear gradient (0=100 mM NaCl buffer, 100%=1500 mM NaCl buffer). The major peak eluting from the affinity column was polymerase. Roughly 60-80 fractions were collected and the column stopped once enzyme eluted.

Each fraction was 3 ml. Aliquots from the fractions were analyzed by SDS-PAGE as indicated in the figures.

Peak fractions were pooled into dialysis tubing, concentrated against solid PEG6000 to 3-4ml then dialysed against Pfe storage buffer; Taq-Cren7 constructs precipitate out if the buffer is not pH 8.5 and 200 mM KCl. It was decided to store all Cren7 fusions in Pfe storage buffer.

6. Extension Time in PCR

This example demonstrates that the Cren7 enhancer domain-DNA polymerase fusions using Sso and Hbu Cren7 enhancer domains amplify larger fragments during PCR compared to unmodified polymerases.

DNA polymerases equivalent to 1.25 u of non-fusion DNA polymerases were tested in an extension efficiency assay by PCR of lambda DNA with a variety of primers.

Lambda DNA was used as the template to assess the relative efficiency of each polymerase in a PCR. For extension efficiency comparison, a set of primers (see Table 4) was used to amplify amplicons of 0.5, 1, 2, 5, 8, 10 and 12 kb in size from the template in a 50 μl reaction. Upon completion of the PCR, 5 μl of the PCR was mixed with loading dye, loaded onto a 1% agarose gel and the gel stained with ethidium bromide.

TABLE 4
Primers used for testing extension efficiency.

| | Amplicon | | SEQ |
|---|---|---|---|
| | Size | | ID |
| Primer | (kb)*¹ | Sequence (5′-3′) | NO: |
| L30350F | - | CCTGCTCTGCCGCTTCACGC | 51 |
| L71-0.5R | 0.5 | TCCGGATAAAAACGTCGATGACATTTGC | 52 |
| L71-1R | 1 | GATGACGCATCCTCACGATAATATCCGG | 53 |
| L72-2R | 2 | CCATGATTCAGTGTGCCCGTCTGG | 54 |
| L72-5R | 5 | CGAACGTCGCGCAGAGAAACAGG | 55 |
| L72-8R | 8 | GCCTCGTTGCGTTTGTTTGCACG | 56 |
| L71-10R | 10 | GCACAGAAGCTATTATGCGTCCCCAGG | 57 |
| L71-12R | 12 | TCTTCCTCGTGCATCGAGCTATTCGG | 58 |

*¹Lambda DNA amplicon size when using L30350F and each R primer.

The PCR reactions contained 20 mM Tris-HCl (pH 8.8), 2 mM MgSO₄, 10 mM KCl (50 mM KCl for the fusion enzymes), 10 mM (NH₄)₂SO₄, 1% Triton® X-100, 200 μM each dNTP, 0.5 μM forward and reverse primers, 130 pg/μl lambda DNA and 1.25 u of enzyme under test.

The cycling protocol was 95° C. for 20 s; 20 cycles of 94° C. for 5 s and 72° C. for 2 min.

The results are shown in FIG. 4 (for Sso KlenTaq fusion) and FIG. 5 (for Hbu Pfu fusion).

It is clear that the Hbu-Pfu fusion was able to amplify all fragments, including the 12 kb fragment. In contrast, Pfu polymerase amplified only up to the 5 kb fragment.

Similarly, the Sso-KlenTaq fusion amplified up to the 8 kb fragments, whereas KlenTaq polymerase amplified only up to the 2 kb fragment with a 2 min extension time.

Thus, the presence of Cren7 enhancer domain in the fusion proteins result in longer amplification products in PCR reactions compared to the unmodified protein.

7. Salt-Tolerance in PCR

The binding of polymerase to a primed DNA template is sensitive to the ionic strength of the reaction buffer due to electrostatic interactions, which is stronger in low salt concentration and weaker in high. This example demonstrates that the Cren7 enhancer domain-DNA polymerase fusions exhibit improved performance in PCR reactions containing elevated KCl concentrations. Without wishing to be bound by theory, it is believed that the presence of the Cren7 enhancer domain in the fusion proteins stabilises the binding interaction of the polymerase to DNA template.

Lambda DNA (130 pg) was used as a template in PCR reactions with primers L30350F and L71-1R (see Table 1 above). The concentration of KCl was varied from 10 mM to 150 mM, while all other components of the reaction buffer were unchanged. The PCR reaction was carried out using a cycling program of 94° C. for 3 min, 20 cycles of 94° C. for 30 s, 55° C. for 30 s, and 72° C. for 30 s, followed by 72° C. for 10 min. Upon completion of the reaction, 5 μl of the PCR reaction products were also analysed in on an agarose gel to verify that amplicons of expected length were generated.

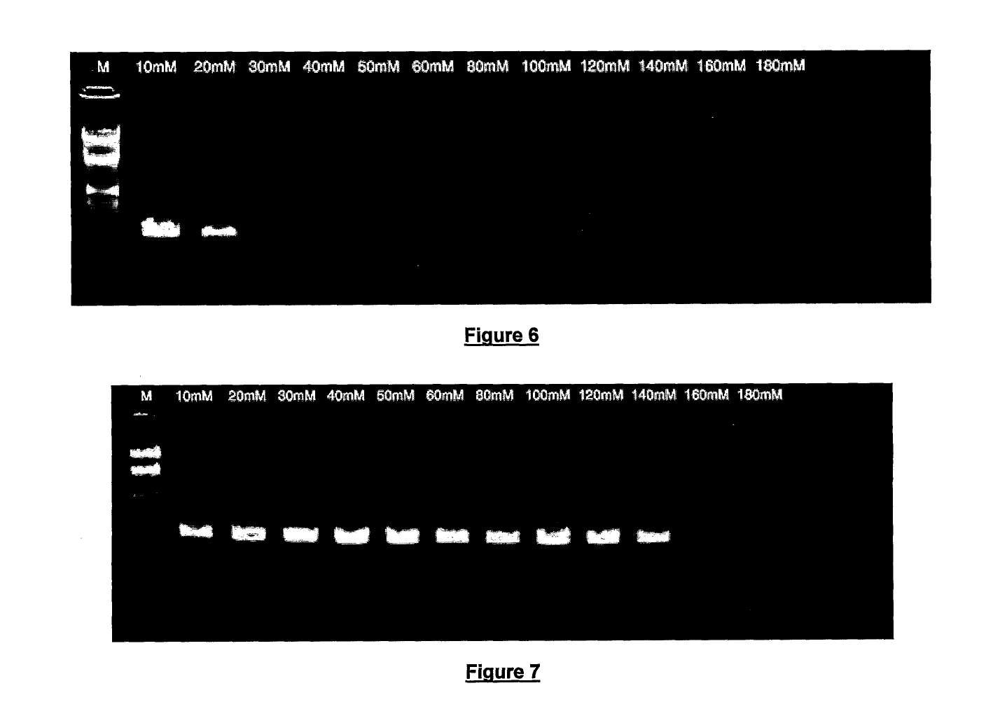

The effects of KCl concentration on the PCR efficiency of Pfu alone versus that of the Ape-Pfu fusion protein, and of KlenTaq alone versus that of the Ape-KlenTaq fusion protein are shown in FIGS. 6, 7, 8 and 9, respectively.

Unmodified Pfu showed a preference for KCl concentration below 20 mM. In contrast, the Ape Cren7 enhancer domain-Pfu fusion protein gave maximum activity in 30-120 mM KCl.

Unmodified KlenTaq showed a preference for KCl concentration below 50 mM. In contrast, the Ape Cren7 enhancer domain-KlenTaq fusion protein gave maximum activity in 30-90 mM KCl.

Thus, the Cren7 enhancer domain-DNA polymerase fusion proteins were more tolerant of elevated KCl concentration in comparison to their counterpart DNA polymerase lacking the Cren7 enhancer domain. This feature of the fusion proteins will allow PCR amplification from low quality of DNA template, e.g., DNA samples prepared from, but not limited to, blood, food and plant sources.

8. Use of Fusion Proteins in qPCR; TagMan® Probe Testing

Quantitative PCR experiments were carried out using the following Taq polymerases:

Wild type Taq polymerase

Chimeric Ape Cren7-Taq polymerase

All enzymes were converted for hotstart by chemical modification with anhydride exactly as described in U.S. Pat. No. 5,677,152. The instrument used for all tests was Cepheid SmartCycler®

A 142 bp mitochondrial target was amplified using the following primers:

Forward:
(SEQ ID NO: 62)
5′ CCA CTG TAA AGC TAA CTT AGC ATT AAC C 3′
Reverse:
(SEQ ID NO: 63)
5′ GTG ATG AGG AAT AGT GTA AGG AGT ATG G 3′

Probe, carrying a FAM fluorescent label at its 5′ end and a TAMRA fluorescent label at its 3′ end:

(SEQ ID NO: 64)
5′ CCA ACA CCT CTT TAC AGT GAA ATG CCC CA 3′

The amplification conditions for wt Taq polymerase were:

50 mM Tris-HCl pH 8.0, 300 nM primers, 100 nM probe, 5 mM MgCl₂, 200 μM dNTPs, 1.25 u DNA polymerase, 30 ng human chromosomal DNA.

The amplification conditions for Ape Cren7-Taq polymerase were:

50 mM Tris-HCl pH 8.0, 80 mM KCl, 300nM primers, 100 nM probe, 5 mM MgCl₂, 200 μM dNTPs, 1.25 u DNA polymerase, 30 ng human chromosomal DNA.

Cycling Conditions:

95° C. for 5 mins initial enzyme activation

45 cycles of:

95° C. 3secs

60° C. variable

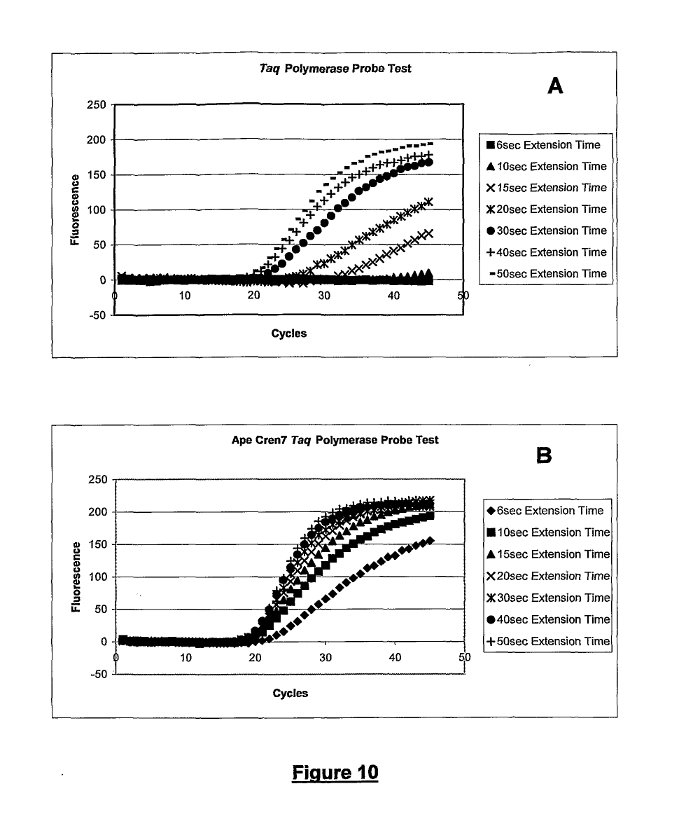

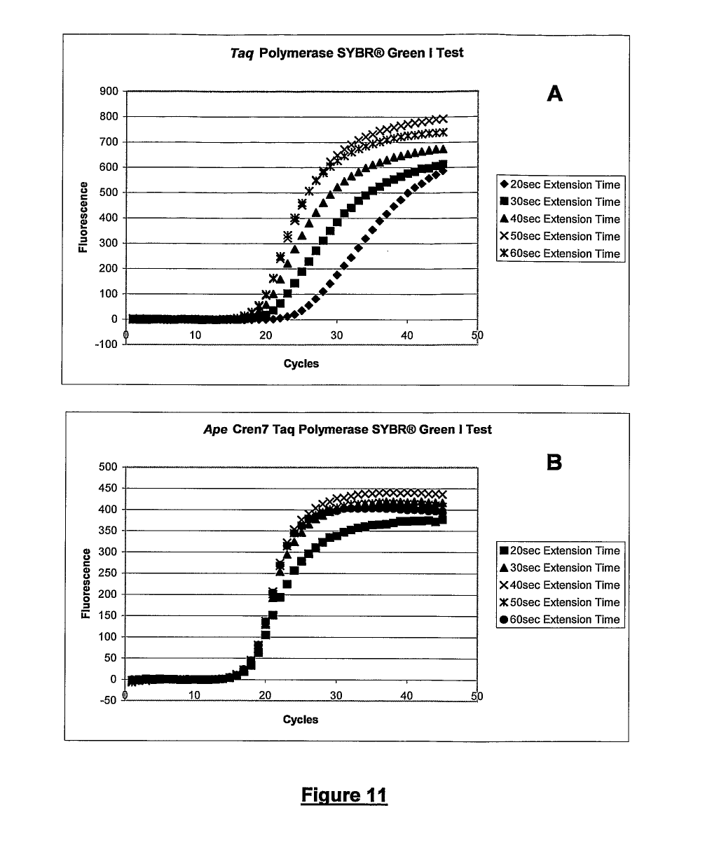

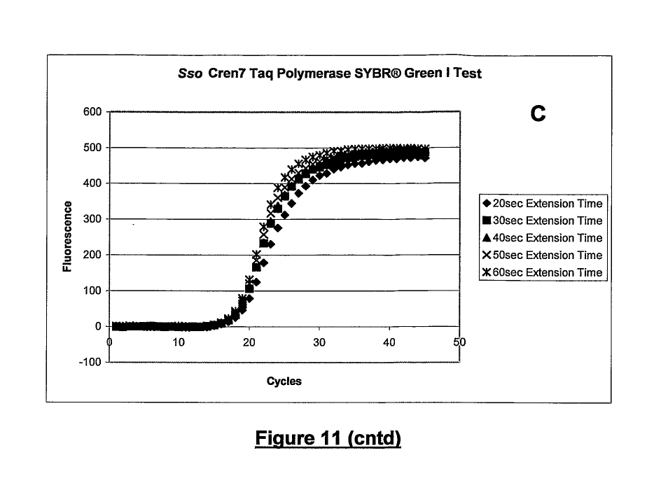

Extension times tested were 50, 40, 30, 20, 15, 10 and 6 seconds. The results in FIG. 10 show that the Cren7 fusion construct required a much lower extension time to gain a similar result, i.e., 10 seconds using the fusion polymerase is equivalent to 30-40 seconds using wild type Taq. This demonstrates that the fusion polymerase has a faster rate of progression than the wild type polymerase.

9. Use of Fusion Proteins in qPCR; SYBR® Green I Testing

Quantitative PCR experiments were carried out using the following Taq polymerases:

Wild type Taq polymerase

Chimeric Ape Cren7-Taq polymerase

Chimeric Sso Cren7-Taq polymerase

All enzymes were converted for hotstart by chemical modification with anhydride exactly as described in U.S. Pat. No. 5,677,152. The instrument used for all tests was Cepheid SmartCycler®

A 142 bp mitochondrial target was amplified using the following primers:

Forward:
(SEQ ID NO: 62)
5′ CCA CTG TAA AGC TAA CTT AGC ATT AAC C 3′
Reverse:
(SEQ ID NO: 63)
5′ GTG ATG AGG AAT AGT GTA AGG AGT ATG G 3′

The amplification conditions for wt Taq polymerase were: 50 mM Tris-HCl pH 8.0, 300 nM primers, 0.5× SYBR® Green I, 5 mM MgCl₂, 200 μM dNTPs, 1.25 u DNA polymerase, 30 ng human chromosomal DNA.

The amplification conditions used for Ape and Sso Cren7-Taq polymerases were:

15 mM Tris-HCl pH 8.3, 80 mM (100 mM for Sso) KCl, 300 nM primers, 0.5×SYBR® Green I, 5 mM MgCl₂, 200 μM dNTPs, 1.25 u DNA polymerase, 30 ng human chromosomal DNA.

Cycling conditions:

95° C. for 5 mins initial enzyme activation

45 cycles of:

95° C. 3 secs

60° C. variable

Extension times tested were 60, 50, 40, 30 and 20 seconds. The results in FIG. 11 show that the Cren7 fusion constructs require a much lower extension time for a similar result, i.e., 20 seconds using the fusion constructs is equivalent to 50 seconds using wild type Taq. The reactions plateau earlier when using the fusion constructs. Again, the results show that the fusion polymerases have a faster rate of progression than the wild type polymerase.

Although the present invention has been described with reference to preferred or exemplary embodiments, those skilled in the art will recognise that various modifications and variations to the same can be accomplished without departing from the spirit and scope of the present invention and that such modifications are clearly contemplated herein. No limitation with respect to the specific embodiments disclosed herein and set forth in the appended claims is intended nor should any be inferred.

All documents cited herein are incorporated by reference in their entirety.

1. A chimeric protein comprising a nucleic acid modifying enzyme domain having nucleic acid modifying activity joined with a Cren7 enhancer domain or variant thereof, in which the Cren7 enhancer domain or variant thereof enhances the activity of the nucleic acid modifying enzyme domain compared with a corresponding protein lacking the Cren7 enhancer domain or variant thereof.

2. A chimeric protein according to claim 1, wherein the Cren7 enhancer domain variant is a functional variant having at least 35% sequence identity with the Cren7 enhancer protein of SEQ ID NO:1.

3. The protein according to claim 1, in which the nucleic acid modifying enzyme domain comprises a nucleic acid polymerase domain.

4. (canceled)

5. (canceled)

6. The protein according to claim 1, in which the Cren7 enhancer domain is one of the group comprising: Sulfolobus solfataricus Cren7 enhancer protein (SEQ ID NO: 1); Sulfolobus acidocaldarius Cren7 enhancer protein (SEQ ID NO: 2); Metallosphaera sedula Cren7 enhancer protein (SEQ ID NO: 3); Staphylothermus marinus Cren7 enhancer protein (SEQ ID NO: 4); Hyperthermus butylicus 0878 Cren7 enhancer protein (SEQ ID NO: 5); Hyperthermus butylicus 1128 Cren7 enhancer protein (SEQ ID NO: 6); Aeropyrum pernix Cren7 enhancer protein (SEQ ID NO: 7); Caldivirga maquilingensis Cren7 enhancer protein (SEQ ID NO: 8); Ignicoccus hospitalis Cren7 enhancer protein (SEQ ID NO: 9); Pyrobaculum islandicum Cren7 enhancer protein (SEQ ID NO: 10); Pyrobaculum arsenaticum Cren7 enhancer protein (SEQ ID NO: 11); Pyrobaculum aerophilum Cren7 enhancer protein (SEQ ID NO: 12); Pyrobaculum calidifontis Cren7 enhancer protein (SEQ ID NO: 13); Thermoproteus neutrophilus Cren7 enhancer protein (SEQ ID NO: 14), Sulfolobus shibatae Cren7 enhancer protein (SEQ ID NO: 59); and Sulfolobus tokodaii Cren7 enhancer protein (SEQ ID NO:60).

7. (canceled)

8. The protein according to claim 1, in which the Cren7 enhancer domain or variant thereof comprises the conserved amino acid sequence: G-X₁-X₂-X₁-X₁-X₃-X₁-P-X₁-K-X₄-W-X₁-L-X₁-P-X₁-G-X₅-X₁-G-V-X₁-X₆-X₇-L-F-X₈-X₁-P-X₉-X₁₀-G-X₁₁X₁-X₁₇ R X₁ X₁ X₁₃ (SEQ ID NO: 15); or comprises the conserved amino acid sequence: X₂-X₁-X₁-X₁-X₁₀-G-X₁-X₁-X₁-X₁-X₃-X₁-P-X₁-K-X₄-W-X₁-L-X₁-P-X₁-G-X₅-X₁-G-V-X₁-X₆-X₇-L-F-X₈-X₁-P-X₉-X₁₀-G-X₁₁-X₁-X₁₂-R-X₁-X₁-X₁₃ (SEQ ID NO:61) where X₁ is any amino acid, X₂ is K, R or E, X₃ is L or no amino acid, X₄ is A, V or T, X₅ is K or R, X₆ is I or V, X₇ is G or A, X₈ is K, R or Q, X₉ is D, N or E, X₁₀ is any or no amino acid, X₁₁ is K or H, X₁₂ is I, V or F, and X₁₃ is I, V or L.

9. The protein according to claim 1, in which the Cren7 enhancer domain is one of the group comprising: Sulfolobus solfataricus Cren7 enhancer protein (SEQ ID NO: 1); Hyperthermus butylicus 1128 Cren7 enhancer protein (SEQ ID NO: 6); Aeropyrum pernix Cren7 enhancer protein (SEQ ID NO: 7) or in which the Cren7 enhancer domain is a functional variant of any of the Cren7 enhancer proteins of SEQ ID NOs: 1, 6 or 7.

10. (canceled)

11. The protein according to claim 3, in which the nucleic acid polymerase domain comprises a thermostable DNA polymerase or a functional mutant, variant or derivative thereof, or of a mesophilic DNA polymerase or a functional mutant, variant or derivative thereof, or of an intermediate temperature DNA polymerase or a functional mutant, variant or derivative thereof.

12. (canceled)

13. The protein according to claim 11, in which the protein is a fusion protein having the sequence of any one of SEQ ID NOs 16-24, or a functional mutant, variant or derivative thereof.

14-17. (canceled)

18. A composition comprising the chimeric protein as defined in claim 1.

19. An isolated nucleic acid encoding the chimeric protein as defined in claim 1.

20. (canceled)

21. A vector comprising the isolated nucleic acid as defined in claim 19.

22. A host cell transformed with the vector of claim 21.

23. A kit comprising the chimeric protein as defined in claim 1, together with packaging materials therefor.

24. A method of modifying a nucleic acid, comprising:
a. contacting the nucleic acid with the chimeric protein as defined in claim 1 under conditions which allow activity of the nucleic acid modifying enzyme domain; and
b. permitting the nucleic acid modifying enzyme domain to modify the nucleic acid.

25. A method of catalysing the synthesis of a polynucleotide from a target nucleic acid, comprising the steps of:
a. providing a chimeric protein as defined in claim 3; and
b. contacting the target nucleic acid with the chimeric protein under conditions which allow the addition by the chimeric protein of nucleotide units to a nucleotide chain using the target nucleic acid, thereby synthesising the polynucleotide.

26. A method of amplifying a sequence of a target nucleic acid using a thermocycling reaction, comprising the steps of:
a. contacting the target nucleic acid with a chimeric protein as defined in claim 3; and
b. incubating the target nucleic acid with the chimeric protein under thermocycling reaction conditions which allow amplification of the target nucleic acid.

27. (canceled)

28. A kit comprising the composition of claim 18, together with packaging materials therefor.

29. A kit comprising the isolated nucleic acid of claim 19, together with packaging materials therefor.

30. A kit comprising the vector of claim 21, together with packaging materials therefor.

31. A kit comprising the host cell of claim 22, together with packaging materials therefor.
